(19) United States
(12) Patent Application Publication
(10) Pub. No.: US 2009/0123514 A1
(43) Pub. Date: May 14, 2009

(54) Electropolymerizable Monomers and Polymeric Coatings on Implantable Devices Prepared Therefrom

(75) Inventor: Abraham J. Domb, Efrat (IL)

Correspondence Address: MARTIN D. MOYNIHAN d/b/a PRTSI, INC., P.O. BOX 16446, ARLINGTON, VA, 22215, US

(73) Assignee: Elutex Ltd., Kiryat-Shmona (IL)

(21) Appl. No.: 11/989,221

(22) PCT Filed: Jul. 19, 2006
(86) PCT No.: PCT/IL2006/000839

§ 371 Date: Jan. 22, 2008

Related U.S. Application Data
(63) Continuation of application No. 11/183,850, filed on Jul. 19, 2005.
Application No. 11/183,850 is a continuation-in-part of application No. 10/148,665, filed on Jun. 3, 2002.
(60) Provisional application No. 60/168,626, filed on Dec. 3, 1999.

(51) Int. Cl. C08F 26/06; A61F 2/02; C07D 207/30; C07D 409/00; C07D 333/02; B05D 3/00; B05D 3/14; B05C 9/08

(52) U.S. Cl. 424/423; 525/326.7; 525/326.8; 548/560; 548/527; 549/29; 427/444; 427/487; 118/620

(57) ABSTRACT

Conductive surfaces of e.g., implantable devices, coated with electropolymerized polymers having active substances attached thereto are disclosed. Electropolymerizable monomers designed and used for obtaining such conductive surfaces and processes, devices and methods for attaching the electropolymerized polymers to conductive surfaces are also disclosed. The polymers, processes and devices presented herein can be beneficially used in the preparation of implantable medical devices.

### FIELD AND BACKGROUND OF THE INVENTION

The present invention relates to conductive surfaces coated with electropolymerized polymers having active substances attached thereto, to electropolymerizable monomers designed and used for obtaining such conductive surfaces and to processes, devices and methods for attaching the electropolymerized polymers to conductive surfaces. The polymers, processes and devices presented herein can be beneficially used in the preparation of implantable medical devices.

In the field of medicine metal structures are often implanted in a living body for various purposes. Such metal structures include, for example, pacemakers, grafts, stents, wires, orthopedic implants, implantable diffusion pumps and heart valves. Implantable metal structures should inherently be characterized by biocompatibility, and more particularly, by both blood and tissue compatibility. An implant is typically considered blood biocompatible when it only mildly induces activation of coagulation factors (e.g., proteins and platelets) and tissue biocompatible when it does not induce excessive cell proliferation and chronic inflammation.

However, the inherent hydrophilic nature of most of the metal surfaces oftentimes adversely affects the biocompatibility of implantable metal structures. Thus, in many applications, upon implantation, the metal surface is eventually covered with a layer of adsorbed biological materials, especially proteins, from the surrounding tissues and fluids. The adsorbed layer of biological material has been implicated in undesired biological reactions including thromboses and inflammations. In addition, pathogenic bacteria, whether directly adhering to the metal surface or attracted by the adsorbed layer, tend to colonize the surface of such devices, turning the devices into the foci of infections. Thus, the hydrophilic nature of the metal surface is the direct cause of the failure of implants. Implant failures are medically harmful, potentially fatal, and more often than not require unpleasant, dangerous and expensive additional surgery.

A number of strategies have been developed for overcoming these problems, the main and common goal thereof being modifying the hydrophilic nature of metal surfaces. Detailed descriptions of these strategies can be found, for example, in U.S. Pat. Nos. 5,069,899, 6,617,142, 4,979,959, 3,959,078, 4,007,089, 5,024,742 and 5,024,742.

One of the most commonly used implantable metal structures is stents. A stent is an endovascular prosthesis which is placed in a peripheral or coronary artery for preventing or treating acute complications of restenosis.

Modification of stents in order to achieve blood and tissue compatibility can be performed by changing the stent material. This, however, oftentimes influences the mechanical behavior of the stent, making it either too rigid or too fragile. Since only the outer layer of the stent interacts directly with the blood and the surrounding tissue, applying a thin coating of a material that can provide the stent surface with the desired biocompatibility is considered a promising strategy.

One strategy for minimizing undesirable biological reactions associated with metal implants such as stents is to coat the metal surface with biomolecules that serve as a substrate for the growth of a protective cell layer. Such biomolecules include, for example, growth factors, cell attachment proteins, and cell attachment peptides. A related strategy involves attachment of active pharmaceutical agents that reduce undesired biological reactions such as antithrombogenics, antiplatelet agents, anti-inflammatories, antimicrobials, and the like, to the metal surface.

A number of approaches have been provided for attaching biomolecules, and other beneficial substances (henceforth collectively termed “active substances”) to metal surfaces of e.g., stents, so as to increase the biocompatibility of the metals.

One approach involves the covalent attachment of a linking moiety to the metal surface, followed by the covalent attachment of the desired active ingredient to the linking moiety. One active ingredient that has been attached to a metal surface by a covalent bond through a linker is the anticoagulant heparin. In the Hepacoat™ stent (Cordis, a Johnson and Johnson company), heparin is covalently bonded to the stent surface. The heparin remains bonded to the stent subsequent to the implantation and the desired effect occurs by interaction in the blood stream.

Another approach involves coating a metal surface with a layer configured to form ionic bonds with an active ingredient. U.S. Pat. No. 4,442,133, for example, teaches a tridodecyl methyl ammonium chloride layer that forms ionic bonds with antibiotic agents. U.S. Pat. No. 5,069,899 teaches a metal surface coated by a layer to which an anionic heparin is attached via an ionic bond.

Another approach involves coating a metal surface with a polymer, and trapping within the polymer an active pharmaceutical ingredient. Once implanted, the active pharmaceutical ingredient diffuses out of the polymer coating causing a desired effect. In the Cypher™ stent (Cordis, a Johnson and Johnson company), for example, the cytostatic Sirolimus (Wyeth Pharmaceuticals) is trapped within a polymer layer coating the stent. Once implanted, the active pharmaceutical ingredient diffuses out of the polymer layer, limiting tissue overgrowth of the stent. The disadvantage of such an implant is that the rate of diffusion of the active pharmaceutical ingredient from the polymer coat is neither controllable nor predictable. Further, this strategy is limited to active pharmaceutical ingredients that can be efficiently entrapped in the polymer yet leach out at a reasonable rate under physiological conditions.

The above technologies, however, are limited by poor adhesion of the coating material to the metal structure; by the rough and non-uniform surface obtained thereby; by a relatively large and uncontrollable thickness of the coat, which may complicate the implantation procedure and performance of the metal structure, and by relatively low flexibility. The latter is particularly significant with respect to stents, which are typically designed as expandable devices. In addition, the current technologies that involve attachment of active substances to the metal surface are mostly associated with uncontrolled release of the active substances in the body.

The above limitations can be overcome by electropolymerization.

Coating conductive surfaces such as metal surfaces using electropolymerizable monomers is highly advantageous since it enables to control the physical and chemical properties of the coated metal surface, by merely controlling parameters of the electrochemical polymerization process such as, for example, the nature of the electrolyte or solvent, current density, and electrode potential. Furthermore, electrocoating is characterized by low processing temperatures that enable formation of highly crystalline deposits with low residual stresses, and the ability to deposit porous surfaces. Electropolymerizable monomers are known in the art and include, for example, anilines, indoles, naphthalenes, pyrroles and thiophenes. When oxidized in the proximity of a surface under electropolymerization conditions, such compounds polymerize to form a polymer film of up to about 15 microns thick. Such a polymer film, although not covalently bonded to the surface, is typically bound to the surface by filling crevices, niches and gaps present in the surface. Such films are widely used in the art as a protective layer for biosensors, as taught, for example, in U.S. Pat. No. 4,548,696.

Implantable medical devices loaded with active substances by means of electropolymerized films have been taught. For example, WO 99/03517, which is incorporated by reference as if fully set forth herein, teaches the ionic bonding of antisense oligonucleotides to a metal surface. In the Journal of Biomedical Materials Research vol. 44, 1999, pp. 121-129 is taught the cationic bonding of heparin to a metal surface. Such an electrostatic binding of the active substance is also limited by uncontrolled release of the active substance upon contacting a living system.

Hence, it is well recognized in the art that modifying the surface of medicinal metal structure, so as to enhance the biocompatibility of such structures and to provide them with further therapeutic characteristics is highly advantageous. The prior art teaches various strategies to overcome the limitations associated with metal implantable devices, which typically involve attachment of active substances either directly or indirectly to a metal surface. The latter include attachment of the active substances to linker molecules or polymers via various chemical interactions (e.g., covalent or ionic bonding, encapsulation, etc.). However, the presently known strategies are limited by poor adhesion of the active substances, the linkers or the polymers to which they are attached, to the metal surface; by a non-uniform coat; by uncontrollable thickness of the coat; by relatively low flexibility; and by uncontrolled release of the active substances.

There is thus a widely recognized need for, and it would be highly advantageous to have, metal surfaces having an active substance attached thereto, devoid of the above limitations, and, particularly, which are a thin, smooth, uniform and flexible and enable a controlled release of the active substance in the body, and can therefore be used for constructing implantable metal structures.

### SUMMARY OF THE INVENTION

According to one aspect of the present invention there is provided an article-of-manufacture comprising: an object having a conductive surface; an electropolymerized polymer being attached to the surface; and at least one active substance being attached to the electropolymerized polymer, provided that the active substance is attached to the polymer via an interaction other than an electrostatic interaction. Also excluded from the scope of the present invention are articles-of-manufacture, and particularly medical devices, in which the active substance is attached to the electropolymerized polymer via covalent interactions, as described in WO 01/39813.

According to further features in preferred embodiments of the invention described below, the object is an implantable device. The implantable device can be a pacemaker, a graft, a stent, a wire, an orthopedic implant, an implantable diffusion pump, an injection port and a heart valve. Preferably, the implantable device is a stent.

According to still further features in the described preferred embodiments the conductive surface comprises stainless steel.

According to still further features in the described preferred embodiments the at least one active substance can be a bioactive agent, a protecting agent, a polymer having a bioactive agent attached thereto, a plurality of microparticles and/or nanoparticles having a bioactive agent attached thereto, and any combination thereof.

According to still further features in the described preferred embodiments the protecting agent can be a hydrophobic polymer, an amphiphilic polymer, a plurality of hydrophobic microparticles and/or nanoparticles, a plurality of amphiphilic microparticles and/or nanoparticles and any combination thereof.

According to still further features in the described preferred embodiments the bioactive agent can be a therapeutically active agent, a labeled agent and any combination thereof. The therapeutically active agent can be an anti-thrombogenic agent, an anti-platelet agent, an anti-coagulant, a growth factor, a statin, a toxin, an antimicrobial agent, an analgesic, an anti-metabolic agent, a vasoactive agent, a vasodilator agent, a prostaglandin, a hormone, a thrombin inhibitor, an oligonucleotide, a nucleic acid, an antisense, a protein, an antibody, an antigen, a vitamin, an immunoglobulin, a cytokine, a cardiovascular agent, endothelial cells, an anti-inflammatory agent, an antibiotic, a chemotherapeutic agent, an antioxidant, a phospholipid, an anti-proliferative agent, a corticosteroid, a heparin, a heparinoid, albumin, a gamma globulin, paclitaxel, hyaluronic acid and any combination thereof.

According to still further features in the described preferred embodiments the active substance is attached to the electropolymerized polymer via an interaction selected from the group consisting of a covalent bond, a non-covalent bond, a biodegradable bond, a non-biodegradable bond, a hydrogen bond, a Van der Waals interaction, a hydrophobic interaction, a surface interaction and any combination thereof.

According to still further features in the described preferred embodiments the active substance is swelled, absorbed, embedded and/or entrapped within the electropolymerized polymer.

According to still further features in the described preferred embodiments the electropolymerized polymer is selected from the group consisting of polypyrrole, polythienyl, polyfuranyl, a derivative thereof and any mixture thereof.

According to still further features in the described preferred embodiments the article-of-manufacture further comprising at least one additional polymer attached to the electropolymerized polymer. The additional polymer can be an electropolymerized polymer and a chemically-polymerized polymer. Preferably, the chemically-polymerized polymer is swelled, absorbed or embedded within the electropolymerized monomer. Also preferably, the chemically-polymerized polymer is covalently attached to the electropolymerized monomer.

According to still further features in the described preferred embodiments the additional polymer forms a part of the electropolymerized polymer.

According to still further features in the described preferred embodiments the active substance is further attached to the additional polymer.

According to still further features in the described preferred embodiments the active substance is attached to the electropolymerized polymer via the additional polymer.

According to still further features in the described preferred embodiments the at least one additional polymer having the active substance attached thereto forms a part of the electropolymerized polymer.

The active substance can also be swelled, absorbed, embedded and/or entrapped within the additional polymer.

According to still further features in the described preferred embodiments the additional polymer can be a hydrophobic polymer, a biodegradable polymer, a non-degradable polymer, a hemocompatible polymer, a biocompatible polymer, a polymer in which the active substance is soluble, a flexible polymer and any combination thereof.

According to still further features in the described preferred embodiments the article-of-manufacture is designed to be capable of controllably releasing the active substance in the body. The releasing is effected during a time period that ranges from about 1 day to about 200 days.

According to still further features in the described preferred embodiments the electropolymerized polymer has a thickness that ranges between 0.1 micron and 10 microns.

According to still further features in the described preferred embodiments the active substance is covalently attached to at least a portion of the electropolymerized polymer.

According to still further features in the described preferred embodiments an amount of the active substance ranges from about 0.1 weight percents to about 50 weight percents of the total weight of the polymer. In one preferred embodiment, the amount of the active substance is about 50 weight percents of the total weight of the polymer.

According to another aspect of the present invention there is provided a process of preparing the article-of-manufacture described herein, the process comprising: providing the object having the conductive surface; providing a first electropolymerizable monomer; providing the active substance; electropolymerizing the electropolymerizable monomer, to thereby obtain the object having the electropolymerized polymer attached to at least a portion of a surface thereof; and attaching the active substance to the electropolymerized polymer.

According to further features in preferred embodiments of the invention described below, the active substance is attached to the electropolymerized polymer via an interaction selected from the group consisting of a covalent bond, a non-covalent bond, a biodegradable bond, a non-biodegradable bond, a hydrogen bond, a Van der Waals interaction, a hydrophobic interaction and a surface interaction.

According to still further features in the described preferred embodiments the active substance is swelled, absorbed, embedded and/or entrapped within the electropolymerized polymer.

According to still further features in the described preferred embodiments attaching of the active substance is performed by: providing a solution containing the active substance; and contacting the object having the electropolymerized polymer attached to at least a portion of a surface thereof with the solution.

According to still further features in the described preferred embodiments the article-of-manufacture further comprises at least one additional polymer attached to the electropolymerized polymer, and the process further comprising: attaching the additional polymer to the electropolymerized polymer, to thereby provide an object having an electropolymerized polymer onto at least a portion of a surface thereof and an additional polymer attached to the electropolymerized polymer.

The additional polymer can be an electropolymerized polymer and the process further comprising: providing a second electropolymerizable monomer; and electropolymerizing the second electropolymerizable monomer onto the object having the electropolymerized polymer onto at least a portion of a surface thereof.

Preferably, the electropolymerizing the second monomer is performed prior to, concomitant with and/or subsequent to attaching the active substance.

The additional polymer can be a chemically-polymerized polymer that is swelled, absorbed or embedded within the electropolymerized monomer, and the process further comprising: providing a solution containing the chemically-polymerized polymer; and contacting the object having the electropolymerized polymer attached to the surface with the solution.

Preferably, the contacting is performed prior to, concomitant with and/or subsequent to attaching the active substance.

The additional polymer can be a chemically-polymerized polymer that is swelled, absorbed or embedded within the electropolymerized monomer, and the process further comprising: providing a solution containing a monomer of the chemically-polymerized polymer; and polymerizing the monomer while contacting the object having the electropolymerized polymer attached to the surface with the solution.

Preferably, the polymerizing is performed prior to, concomitant with and/or subsequent to attaching the active substance. Also preferably, the chemical polymerization is performed prior to, concomitant with and/or subsequent to attaching the active substance.

The additional polymer can be a chemically-polymerized polymer that forms a part of the electropolymerized polymer and providing the first electropolymerizable monomer comprises providing a first electropolymerizable monomer having a functional group capable of interacting with or forming the additional polymer.

Preferably, the functional group is selected capable of forming the additional polymer, the process further comprising: subjecting the object having the electropolymerized polymer attached thereto to a chemical polymerization of the functional group.

Also preferably, the functional group is selected capable of participating is the formation of the additional polymer and the process further comprising: providing a solution containing a substance capable of forming the additional polymer; and contacting the object having the electropolymerized polymer attached to the surface with the solution.

Preferably, the contacting is performed prior to, concomitant with and/or subsequent to attaching the active substance. Also preferably, the functional group is selected from the group consisting of a photoactivatable group, a cross-linking group and a polymerization-initiating group.

According to further features in preferred embodiments of the invention described below, the electropolymerizable monomer and/or the electropolymerizing is selected so as to provide an electropolymerized polymer having a thickness that ranges between 0.1 micron and 10 microns. The electropolymerizable monomer can be an N-alkyl pyrrole derivative in which the alkyl has at least 3 carbon atoms.

According to further features in preferred embodiments of the invention described below, the active substance is covalently attached to at least a portion of the electropolymerized polymer, the electropolymerizable monomer has the active substance covalently attached thereto and the attaching the active substance to the electropolymerized polymer is effected by electropolymerizing the monomer.

According to further features in preferred embodiments of the invention described below, the active substance is covalently attached to at least a portion of the electropolymerized polymer, and providing the first electropolymerizable monomer comprises providing a first electropolymerizable monomer having a reactive group capable of covalently attach the active substance.

Attaching the active substance can comprise reacting a solution containing the active substance with the object having the electropolymerized polymer attached to at least a portion of a surface thereof.

According to further features in preferred embodiments of the invention described below, the process further comprising, prior to the electropolymerizing, treating the surface of the object so as to enhance the adhesion of the electropolymerized polymer to the surface.

The treating can comprise: manually polishing the surface; and rinsing the surface with an organic solvent.

Alternatively, the treating can also comprise: contacting the surface with an acid (e.g., nitric acid); rinsing the surface with an aqueous solvent; and subjecting the surface to sonication.

Further alternatively, the treating can further comprise: subjecting the surface to sonication; and rinsing the surface with an organic solvent, an aqueous solvent or a combination thereof.

Preferably, the sonication is performed in the presence of carborundum.

Also preferably, the sonication is performed in an organic solvent.

According to yet another aspect of the present invention there is provided an electropolymerizable monomer having one or more of the following functional groups: (i) a functional group capable of enhancing an adhesion of an electropolymerized polymer formed from the electropolymerizable monomer to a conductive surface; (ii) a functional group capable of enhancing absorption, swelling or embedding of an active substance within an electropolymerized polymer formed from the electropolymerizable monomer; (iii) a functional group capable of forming a chemically-polymerized polymer; (iv) a functional group capable of participating in the formation of a chemically-polymerized polymer; (v) a functional group capable of providing an electropolymerized polymer formed from the electropolymerizable monomer having a thickness that ranges from about 0.1 micron to about 10 microns; (vi) a functional group capable of enhancing the flexibility of an electropolymerized polymer formed from the electropolymerizable monomer; and (vii) a functional group capable of covalently attaching an active substance thereto.

According to further features in preferred embodiments of the invention described below, the functional group capable of enhancing an adhesion of an electropolymerized polymer formed from the electropolymerizable monomer to a conductive surface, group capable of enhancing an absorption, swelling or embedding of an active substance within an electropolymerized polymer formed from the electropolymerizable monomer, capable of covalently attaching an active substance thereto and/or capable of providing an electropolymerized polymer formed from the electropolymerizable monomer having a thickness that ranges from about 0.1 micron to about 10 microns is an ω-carboxyalkyl.

According to further features in preferred embodiments of the invention described below, the electropolymerizable monomer can be a pyrrole having the functional group is attached thereto.

According to further features in preferred embodiments of the invention described below, the alkyl has at least 3 carbon atoms.

According to further features in preferred embodiments of the invention described below, the functional group capable of enhancing the flexibility of an electropolymerized polymer formed from the electropolymerizable monomer is a polyalkylene glycol or a derivative thereof.

According to an additional aspect of the present invention there is provided an electropolymerizable monomer comprising at least two electropolymerizable moieties being linked to one another.

The at least two electropolymerizable moieties can be the same or different. Each of the electropolymerizable moieties is preferably independently selected from the group consisting of substituted or unsubstituted pyrrole, thienyl and furanyl.

The at least two electropolymerizable moieties can be linked to one another via a covalent bond, a spacer or a combination thereof.

The spacer is preferably selected from the group consisting of a substituted or unsubstituted alkyl, a substituted or unsubstituted alkenyl, a substituted or unsubstituted alkynyl, a substituted or unsubstituted cycloalkyl, and a substituted or unsubstituted polyalkylene glycol.

According to yet an additional aspect of the present invention there is provided an electropolymerizable monomer comprising at least one electropolymerizable moiety and at least one functional group capable of forming a chemically-polymerized polymer being attached to the electropolymerizable moiety.

According to still an additional aspect of the present invention there is provided an electropolymerizable monomer comprising at least one electropolymerizable moiety and at least one functional group capable of participating in the formation of a chemically-polymerized polymer being attached to the electropolymerizable moiety. The functional group can be, for example, a photoactivatable group and a cross-linking group.

According to further features in preferred embodiments of the invention described below, the functional group capable of forming a chemically-polymerized polymer can be an allyl group and a vinyl group.

According to further features in preferred embodiments of the invention described below, the functional group capable of participating in the formation of a chemically-polymerized polymer can be a photoactivatable group and a cross-linking group.

According to still another aspect of the present invention there is provided a method of treating a conductive surface so as to enhance the adhesion of an electropolymerized polymer to the surface, which comprises subjecting the surface, prior to forming the electropolymerized polymer thereon, to at least one procedure selected from the group consisting of manually polishing the surface, contacting the surface with nitric acid, subjecting the surface to sonication and any combination thereof.

According to further features in preferred embodiments of the invention described below, the sonication is performed in the presence of carborundum.

According to an additional aspect of the present invention there is provided a device for holding a medical device while being subjected to electropolymerization onto a surface thereof, the device comprising a perforated encapsulation, adapted to receive the medical device, and at least two cups adapted for enabling electrode structures to engage with the perforated encapsulation hence to generate an electric field within the perforated encapsulation.

According to further features in preferred embodiments of the invention described below, the perforated encapsulation is designed and constructed to allow fluids and chemicals to flow therethrough.

According to further features in preferred embodiments of the invention described below, the at least one medical device comprises at least one stent assembly.

According to yet an additional aspect of the present invention there is provided a cartridge, comprising a plurality of holding devices, as described herein, and a cartridge body adapted for enabling the plurality of holding devices to be mounted onto the cartridge body.

According to further features in preferred embodiments of the invention described below, the cartridge comprises at least 3 holding devices.

According to still an additional aspect of the present invention there is provided a system for coating at least one medical device, the system comprising in operative arrangement, at least one holding device, as described herein, a conveyer and a plurality of treating baths arranged along the conveyer, wherein the conveyer is designed and constructed to convey the at least one holding device such that the at least one holding device is placed within each of the plurality of treating baths for a predetermined time period and in a predetermined order.

According to further features in preferred embodiments of the invention described below, the system further comprises a cartridge having a cartridge body adapted for enabling the at least one holding device to be mounted onto the cartridge body.

According to further features in preferred embodiments of the invention described below, the perforated encapsulation is designed and constructed to allow fluids and chemicals to flow therethrough.

According to further features in preferred embodiments of the invention described below, the plurality of treating baths comprises at least one electropolymerization bath and at least one active substance solution bath. At least one of the plurality of treating baths can be a pretreatment bath, a washing bath, a rinsing bath and a chemical polymerization bath.

Preferably, the electropolymerization bath comprises at least one electrode structure, mounted on a base of the electropolymerization bath and connected to an external power source.

Additionally, the conveyer is operable to mount the at least one holding device on the at least one electrode structure, thereby to engage the at least one electrode structure with a first side of the perforated encapsulation.

According to further features in preferred embodiments of the invention described below, the system further comprises an arm carrying at least one electrode structure and operable to engage the at least one electrode structure with a second side of the perforated encapsulation.

The present invention successfully addresses the shortcomings of the presently known configurations by providing novel processes for coating metal surfaces, which result in stable, uniform and adherent coatings and may further be designed to controllably release active substances that are attached thereto.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” or “process” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “amine” describes both a —NR′R″ group and a —NR′-group, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

The terms “alkyl” and “alkylene” describe a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. The alkyl group may be substituted or unsubstituted.

The terms “alkenyl” and “alkenylene” describe an alkyl, as defined herein, having at least two carbon atoms and at least one double bond.

The term “vinyl” describes a —HC═CH₂ group.

The term “allyl” describes a —CH₂CH═CH₂ group.

The terms “alkynyl” and “alkynylene” describe an alkyl, as defined herein, having at least two carbon atoms and at least one triple bond. An example is acetylene —CH≡CH.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted. Substituted cycloalkyl may have one or more substituents.

The term “aryl” describes an all-carbon monocyclic or fused ring polycyclic (i.e., rings which share adjacent pairs of carbon atoms) groups having a completely conjugated pi-electron system. The aryl group may be substituted or unsubstituted. Substituted aryl may have one or more substituents.

The term “halide” which is also referred to herein interchangeably as “halo” describes fluorine, chlorine, bromine or iodine.

The term “haloalkyl” describes an alkyl group as defined above, further substituted by one or more halide.

The term “sulfate” describes a —O—S(═O)₂—OR′ or a —O—S(═O)₂—O-group, where R′ is as defined hereinabove.

The term “sulfonate” describes a —S(═O)₂—R′ group or a —S(═O)₂— group, where R′ is as defined herein.

The term “disulfide” refers to a —S—SR′ group or a —S—S— group, where R′ is as defined herein.

The term “phosphonate” describes a —P(═O)(OR′)(OR″) group or a —P(═O)(OR′)(O)— group, with R′ and R″ as defined herein.

The term “carbonyl” or “carbonate” as used herein, describes a —C(═O)—R′ group or a —C(═O)— group, with R′ as defined herein.

The term “hydroxyl” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” or “thiol” describes a —SH group.

The term “acyl halide” describes a —C═O)R″″ group wherein R″″ is halide, as defined hereinabove.

The term “carboxylate” or “carboxy” describes a —C(═O)—OR′ group or a —C(═O)—O— group, where R′ is as defined herein.

The term “acrylate” describes a CH₂═CR″—C(═O)R′, with R′ and R″ as defined herein. The term “acrylamide” describes a CH₂═CR″—C(═O)NR′R′″, with R′ and R″ as defined herein and R′″ being as defined for R′.

As used herein, the singular form “a,” “an,” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration of an electropolymerization setup, according to preferred embodiments of the present invention, whereby the coating on the metallic surface is conducted in a solution comprising the desired monomer/s and a buffer, through the application of current, whereby the metallic surface (stent) acts as an anode;

FIG. 2 is a schematic representation of a step electropolymerization process of pyrrole, wherein a monomer is first activated by current to obtain an active radical, which then reacts with other pyrrole radical in a coupling reaction;

FIG. 3 is a schematic illustration of a stent having protective functional groups attached to its surface;

FIG. 4 is a schematic illustration of a stent having a drug and/or a drug entrapped in a polylactic acid particle (PLA) attached to its surface, wherein the drug can be controllably released from the stent;

FIG. 5 is a schematic illustration of a stent having a drug (D) attached thereto, wherein the drug is active while being bound to the stent;

FIG. 6 presents the chemical structure of exemplary electropolymerizable monomers possessing a reactive side chain, according to preferred embodiments of the present invention (R and R′ represent organic residues and Y represents a degradable or non-degradable chemical bond);

FIGS. 7(A-B) present the chemical structure of exemplary electropolymerizable monomers having a drug or nanoparticles encapsulating a drug covalently attached thereto (FIG. 7A), and an exemplary electropolymerized polymer obtained therefrom (FIG. 7B);

FIG. 8 is a typical cyclic voltametry diagram of electropolymerization of pyrrole derivatives, according to preferred embodiments of the present invention;

FIG. 9 presents comparative plots demonstrating the effect of the number of CV on the thickness of electropolymerized polypyrrole derivatives according to the present embodiments;

FIGS. 10(A-J) are SEM micrographs of surfaces of stainless steel plates coated with various electropolymerized pyrrole derivatives;

FIG. 11 presents plots demonstrating the release profile of Paclitaxel incorporated in electropolymerized poly(butyl ester)pyrrole with (1) and without (2) PLA;

FIG. 12 presents a plot demonstrating the release profile of Paclitaxel embedded in an exemplary electropolymerized polypyrrole-coated stent according to the present embodiments;

FIG. 13 presents a plot demonstrating the release profile of Paclitaxel embedded in an exemplary electropolymerized polypyrrole and PLA-coated stent according to the present embodiments;

FIG. 14 is a schematic representation of an exemplary holding device, according to the present embodiments;

FIG. 15 is a schematic representation of an exemplary cartridge according to the present embodiments; and

FIG. 16 is a schematic representation of an exemplary system, according to the present embodiments.

### DESCRIPTION OF THE PREFERRED EMBODIMENTS

The present invention is of novel coatings of conductive surfaces, which are capable of efficiently incorporating therein various active substances that may provide the surface with added therapeutic value and/or with enhanced biocompatibility. The novel coatings described herein can thus be beneficially used as coatings of medical devices, and in particular of implantable devices.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As is discussed hereinabove, the use of medical devices which have a metal surface is often limited by their hydrophilic nature, which leads to undesired reactions (e.g., thrombosis and inflammation) and adversely affects the biocompatibility of the device. Strategies developed to improve the biological performance of such devices include coating the metal surface by a hydrophobic layer, which may optionally further include a bioactive agent (e.g., a drug). While the prior art teaches various methods of attaching hydrophobic moieties to metal surfaces, these methods are typically limited by poor adhesion of the coating and/or uncontrolled release of the bioactive agents therefrom.

Among metals, stainless steel is of special importance due to its wide use in orthopedic implants and other implantable medical devices, owing to its corrosion resistance and superior mechanical properties. The biocompatibility of stainless steel implants can be significantly improved by modifying its surface with organic molecules or polymers. With the increased interest in drug eluting medical devices in general and stents in particular, where metallic surfaces are coated with a drug-loaded polymer, adherent and uniform thin coatings (1-2 μm) are desired.

However, the presently used technologies and particularly methods for coating devices by means of dipping or spraying a polymer solution, are limited by poor adhesion of the coating material to the metal structure; by the rough and non-uniform surface obtained thereby; by a relatively large and uncontrollable thickness of the coat (about 15-20 μm), which may complicate the implantation procedure and performance of the metal structure, and by relatively low flexibility. The latter is particularly significant with respect to stents, which are typically designed as expandable devices. Furthermore, some of the known biopolymers used for coating medical devices, such as polyurethane, polyacrylates and various lipids and phospholipid derivatives, are oftentimes incompatible with the implant environment, blood components and tissue. In addition, the current technologies that involve attachment of active substances to the metal surface are mostly associated with uncontrolled release of the active substances in the body.

As is further discussed hereinabove, the above limitations can be overcome by electropolymerization. Coating of conductive polymers on metal surfaces using electrochemical polymerization which provides stable, adherent and strong electro-conducting coatings have been extensively used in the field of biosensors. As described above, various active enzymes have been conjugated to the tip of biosensors via electrochemical polymerization of conducting monomers including pyrrole, carbazole, and thiophene. This coating indeed adheres well to the metallic tip and is used as conducting polymer capable of transfer of the current signals generated by the enzyme attached to the polymer when activated.

Although a significant work was conducted on the synthesis of various conducting polymers for use in biosensors very little was reported on the use of suitable electropolymerizable reactive coatings for medical devices.

The present invention overcomes the limitations associated with the presently known metallic medical devices by providing novel methodologies for coating metallic surfaces. These methodologies involve deposition of an electropolymerized polymeric film, which retains its consistency and adhesiveness while in the body of a patient and thus fulfills the safety and efficacy requirements for coating of implantable devices. These methodologies further involve the incorporation of active substances in the polymeric coating, which may provide, in addition to improved biocompatibility, an added value to the device performance in terms of its therapeutic effect and/or the mechanical and/or physical characteristics of the device. When therapeutically active substances are incorporated in the coating, the methodologies described herein enable to design coatings that would enable the slow release of the substance in a controlled manner. The active substances may be incorporated in the polymeric coating by various interactions (e.g., covalent, hydrogen bonds, swelling, absorption and the like), depending on the desired rate and nature of their release.

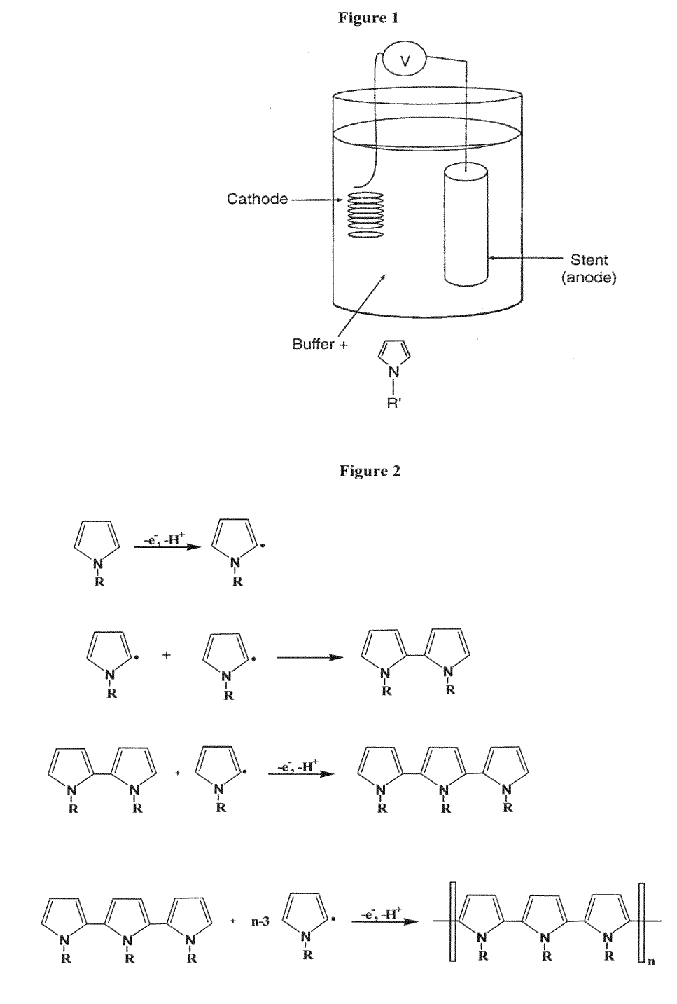

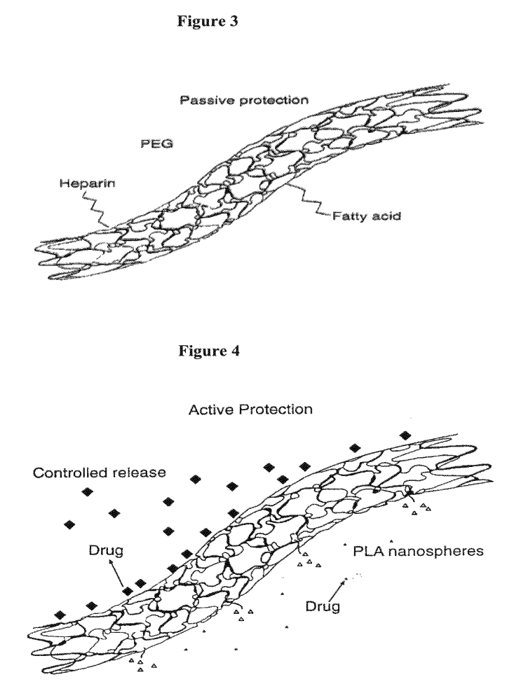

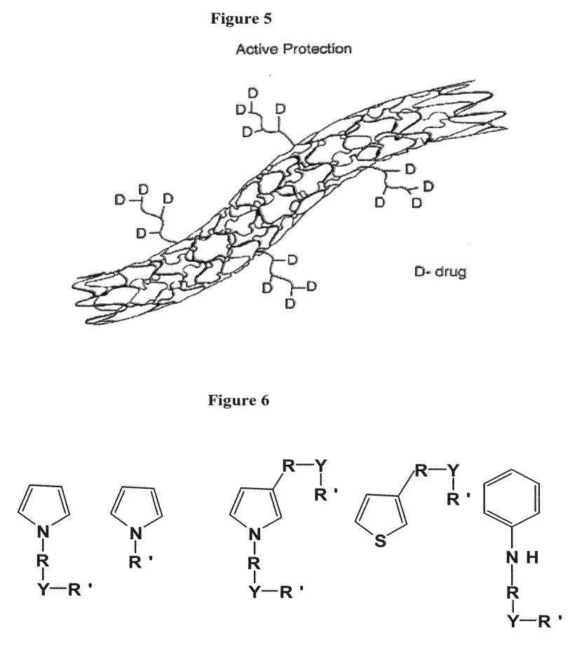

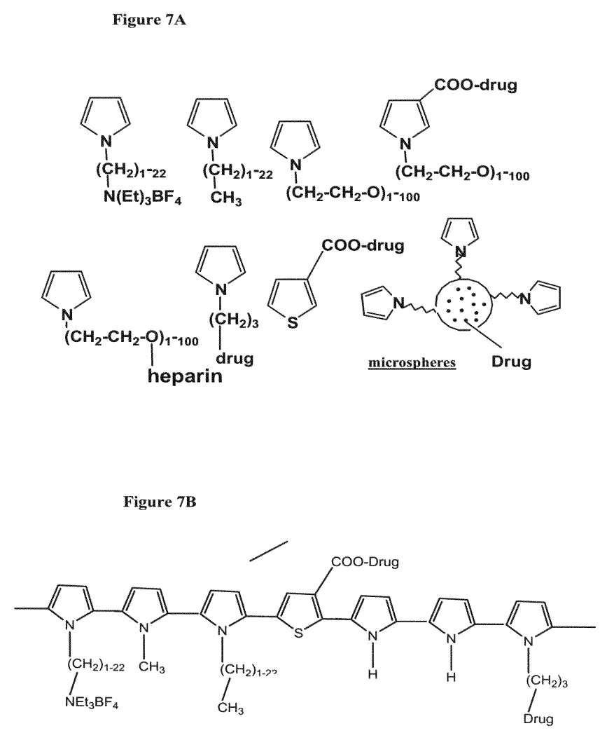

The present invention is thus of forming adherent coatings onto metallic surfaces, which are capable of being loaded with an active substance and release the substance, if desired, during periods of one day to several months in a controlled manner. These adherent, well-fitted onto metal structure, strong and stable coatings are prepared by polymerizing oxidizable monomers onto a metal surface by electropolymerization (see, FIG. 1, for an exemplary electropolymerization). The preferred oxidizable monomers are pyrrole derivatives and pyrrole oligomers possessing affinity to metal surfaces upon electropolymerization onto metal surface. The chemical chain-reactions leading to the electropolymerization of pyrroles are depicted in FIG. 2. These coating can be used as is for drug loading and release over time, or may serve as a platform to embed within the coating or onto the coating a layer of another polymer either by secondary polymerization of reactive monomer units absorbed into the electropolymerization coating or attached to the coating via a chemical bond or specific interaction, as is schematically illustrated in FIGS. 3-5.

While reducing the present invention to practice, as described, for example, in WO 01/39813 and in U.S. patent application Ser. No. 10/148,665, which are incorporated by reference as if fully set forth herein, a range of newly synthesized electrochemically polymerizable monomers, which are also referred to as electropolymerizable monomers, have been designed and successfully prepared. These electropolymerizable monomers were designed capable of attaching bioactive agents and other substances thereto either prior to or after electropolymerization. Particularly, such electropolymerizable monomers which have functional groups that enable to covalently attach thereto an active substance, either per se or as a part of a carrier entity (e.g., polymers and micro- and nanoparticles), have been prepared. The electropolymerizable monomers were designed such that the active substance is attached thereto via covalent interactions, which are either biodegradable or non-degradable, such that a slow release of the active substance is enabled in a controlled manner.

Thus, the following electropolymerizable monomers have been prepared: (i) electropolymerizable monomers to which a bioactive agent is covalently attached via a cleavable, biodegradable bond such as an ester, amide, imine; (ii) electropolymerizable monomers to which the active agent is covalently attached via a spacer; (iii) micro- and nano-particles incorporating active agents and further containing electropolymerizable groups; (iv) electropolymerizable monomers having a polymer attached thereto, which provides for passive protection of the coated surface and further enables the incorporation of an active agent therein.

The various electropolymerizable monomers were used to provide a stable polymeric coating that is biocompatible and biostable. The various electropolymerizable monomers were further used to provide a thin adherent and uniform coating. The various electropolymerizable monomers were further deigned to release the active agents in a controlled manner to the surrounding tissue for local delivery and action.

Thus, the electropolymerizable monomers were designed such that a polymeric coating with predetermined characteristics, which provides for improved short and long term performance of implantable devices such as stents in the body cavities, could be obtained.

While further reducing the present invention to practice, electropolymerizable monomers, designed such that a polymeric film in which active agents can be embedded would be obtained upon electropolymerization thereof, have been prepared. Thus, non-covalently attached active substances can be incorporated, for example, in an insoluble, three dimensional, crosslinked matrix in film form and controllably-released therefrom.

Thus, as is demonstrated in the Examples section that follows, various derivatives of electropolymerizable monomers have been designed, prepared and used for preparing polymeric coatings deposited on metal surfaces. The electropolymerizable monomers were designed such that active substances (e.g., drugs and protecting agents) would be incorporated in the resulting polymeric coatings and could be controllably released over time, if desired. The electropolymerizable monomers were further designed such that active substances would be incorporated in the resulting polymeric coatings via either covalent or non-covalent interactions.

These newly designed electropolymerized polymeric coatings of the present invention can be, for example:

(i) polymers of certain N-alkyl pyrrole monomers which exhibit specific affinity to metal surfaces and remain intact even after expansion such as in the case of an expandable stent;

(ii) polymers of pyrrole derivatives with reactive side groups such as vinyl, amino, alcohol or carboxylic acids that can further bind a polymer or a molecule of interest or initiate polymerization of a reactive monomer; and

(iii) polymers of pyrrole derivatives that form a porous thin coating suitable for embedding another polymer to form an interpenetrating system. The second polymer can be loaded into the primer porous polypyrrole coating, or monomers that upon activation polymerize into an interpenetrating polymer system can be loaded.

Thus, according to one aspect of the present invention, there is provided an article-of-manufacture which comprises: an object having a conductive surface; an electropolymerized polymer being attached to the surface; and at least one active substance being attached to the electropolymerized polymer. The active substance is attached to the polymer via non-covalent interactions whereby articles-of-manufacture in which the active substance is attached to the polymeric coating via electrostatic interactions are excluded from the scope of the invention. Further excluded from the scope of the present invention are articles-of-manufacture in which the active substance is attached to the polymeric coating via covalent interactions, and particularly, the electropolymerizable monomers, polymers prepared therefrom and devices containing these polymers, which are described in WO 01/39813 and in U.S. patent application Ser. No. 10/148,665.

As used herein, the phrase “electrostatic interactions” refers to interactions that are formed between two substances that have opposite charges, namely, a positively charged substance and a negatively charged substance. Such interactions typically involve ionic bonds.

As discussed in detail hereinabove, attachment of active substances to implantable devices by electrostatic interactions is limited by the uncontrolled release thereof.

While, as is discussed hereinabove, modifying a hydrophilic metal surface of an object is highly beneficial in medical devices, particularly implantable medical devices, the object is preferably a medical device. The medical device can be any metal device that comprises a metal surface and include, for example, extra corporeal devices such as apheresis equipment, blood handling equipment, blood oxygenators, blood pumps, blood sensors, fluid transport tubing and the like. However, modifying a hydrophilic metal surface is particularly useful in implantable medical devices such that the medical device can be an intra corporeal device such as, but not limited to, aortic grafts, arterial tubing, artificial joints, blood oxygenator membranes, blood oxygenator tubing, bodily implants, catheters, dialysis membranes, drug delivery systems, endoprostheses, endotracheal tubes, guide wires, heart valves, intra-aortic balloons, medical implants, pacemakers, pacemaker leads, stents, ultrafiltration membranes, vascular grafts, vascular tubing, venous tubing, wires, orthopedic implants, implantable diffusion pumps and injection ports.

Particularly preferred medical devices according to the present invention are stents, and expandable stents in particular. Such stents can be of various types, shapes, applications and metal compositions and may include any known stents. Representative examples include the Z, Palmaz, Medivent, Strecker, Tantalum and Nitinol stents.

The phrase “implantable device” is used herein to describe any medical device that is placed within a body cavity for a prolonged time period.

Suitable conductive surfaces for use in the context of the present invention include, without limitation, surfaces made of one or more metals or metal alloys. The metal can be, for example, iron, steel, stainless steel, titanium, nickel, tantalum, platinum, gold, silver, copper, any alloys thereof and any combination thereof. Other suitable conductive surfaces include, for example, shape memory alloys, super elastic alloys, aluminum oxide, MP35N, elgiloy, haynes 25, stellite, pyrolytic carbon and silver carbon.

Since particularly useful objects are implantable medical devices, and further since such devices are typically made of stainless steel, the conductive surface preferably comprises stainless steel.

As is discussed in detail hereinabove, medical devices having metal surfaces in general and stainless steel surfaces in particular suffer many disadvantages, mostly due to the poor blood and/or tissue biocompatibility of such surfaces. As is further discussed hereinabove, poor blood biocompatibility typically results in activation of coagulation proteins and platelets whereby poor tissue biocompatibility typically results in excessive cell proliferation and inflammation. Modifying the surface so as to enhance its biocompatibility can be performed by chemical and/or physical means that are aimed at improving the surface characteristics in terms of charge, wettability and topography. These can be achieved by attaching to surface a thin layer (film) of substances such as polymers (e.g., poly(ethylene glycol), Teflon and polyurethane). Alternatively, modifying the surface can be performed by attaching a bioactive agent to the surface, which can reduce the adverse effects associated with the poor biocompatibility or can induce additional beneficial effects.

Thus the conductive surface, according to the present invention, has one or more active substances being attached to the electropolymerized polymer.

The phrase “active substance” is used herein to describe any substance that may beneficially affect the characteristics of the object's surface (e.g., the biological, therapeutic, chemical and/or physical characteristics of the surface) and includes, for example, substances that affects the charge, wettability, and/or topography of the surface, substances that reduce the adverse side effects induced by the surface and/or therapeutically active agents that may provide the object with additional therapeutic effect.

Hence, preferred active substances, according to the present invention, include, without limitation, bioactive agents, protecting agents, polymer having a bioactive agent attached thereto, microparticles and/or nanoparticles having a bioactive agent attached thereto, and any combination thereof.

As used herein, the phrase “protecting agent” describes an agent that can protect the coated surface from undergoing undesired reactions and thus can render the object relatively inert regarding undesired interactions with its environment. Thus, when the object is an implantable device, a protecting agent can prevent or reduce undesired absorption of biological materials such as proteins, from the surrounding tissues and fluids, which may lead to thromboses and inflammations.

Since, as described hereinabove, most of the undesired interactions associated with implantable devices results from the hydrophilic nature of metal surfaces, preferred protecting agents that are suitable for use in the context of the present invention are hydrophobic or amphiphilic substances, and, more particularly, hydrophobic or amphiphilic substances such as polymers, microparticles and nanoparticles.

Exemplary polymers that are suitable for use as protecting agents in the context of the present invention include, without limitation, non-degradable polymers such as polyethylene glycols (PEGs, having MW in the range of 100-4000), and substituted polyethylene glycols and analogs thereof (e.g., Jeffamine), as well as polymers formed by electropolymerization of alkylated electropolymerizable monomers, wherein the alkyl has more than 5, preferably more than 10 carbon atoms.

Exemplary particles that are suitable for use in this context of the present invention include non-degradable microparticles and/or nanoparticles, which can be formed from various substances and via various synthetic routes well known in the art.

Thus, polymers and particles such as nanoparticles and microparticles can be applied per se onto a surface, so as to affect its characteristics, as described hereinabove. Bioactive agents are applied so as to affect the surface's biological characteristics, and particularly, its therapeutic activity. Polymers and particles having a bioactive agent attached thereto are typically applied onto a surface so as to affect its physical and chemical characteristic and on the same time to act as carriers of one or more bioactive agents.

Polymers and particles that serve as carriers of a bioactive agent can be either stable or biodegradable when applied. The term “biodegradable” is used to describe such materials that may be decomposed upon reaction with e.g., enzymes (hydrolases, amidases, and the like), whereby the term “stable” is used to describe such materials that remain intact when applied, at least for a prolonged time period. The release of the bioactive agent from a stable carrier is typically performed by diffusion of the agent.

The phrase “having a bioactive agent being attached thereto” with respect to polymers, particles and any other moiety mentioned herein, is used to describe any form in which the bioactive agent is attached to the moiety and therefore includes covalent attachment, by either biodegradable bonds or stable bonds, encapsulation, swelling, absorption and any other acceptable attachment form.

The phrase “bioactive agent” is used herein to describe an agent capable of exerting a beneficial activity in a subject. Such a beneficial activity include, as is discussed hereinabove, reducing adverse side effects induced by the surface and/or any other therapeutic activity, depending on the desired application of the object.

The bioactive agent can therefore be a therapeutically active agent, which is also referred to herein interchangeably as a pharmaceutically active agent, an active pharmaceutical agent or simply an active agent.

The bioactive agent can further be a labeling agent, which may serve for detecting and/or locating the substance to which it is attached in the body and may be used, for example, for diagnosis and follow-up purposes.

The phrase “labeling agent” is therefore used herein to describe a detectable moiety or a probe and includes, for example, chromophores, fluorescent compounds, phosphorescent compounds, heavy metal clusters, and radioactive labeling compounds, as well as any other known detectable moieties.

In some cases, the therapeutically active agent may be labeled and thus further serves as a labeling agent. Similarly, some labeling agents, such as radioisotopes, can also serve as therapeutically active agents.

The bioactive agent can be selected according to the desired application of the object. In cases where the object is a medical device, the bioactive agent is selected depending on the condition being treated by the medical device and the bodily cavity in which the device is implanted.

Representative examples of bioactive agents which are suitable for use in the context of the present invention, namely, for being incorporated within the polymeric coating include, without limitation, anti-thrombogenic agents, anti-platelet agents, anti-coagulants, statins, toxins, growth factors, antimicrobial agents, analgesics, anti-metabolic agents, vasoactive agents, vasodilator agents, prostaglandins, hormones, thrombin inhibitors, oligonucleotides, nucleic acids, antisenses, proteins (e.g., plasma proteins, albumin, cell attachment proteins, biotin and the like), antibodies, antigens, vitamins, immunoglobulins, cytokines, cardiovascular agents, endothelial cells, anti-inflammatory agents (including steroidal and non-steroidal), antibiotics (including antiviral agents, antimycotics agents and the like), chemotherapeutic agents, antioxidants, phospholipids, anti-proliferative agents, corticosteroids, heparins, heparinoids, albumin, gamma globulins, paclitaxel, hyaluronic acid and any combination thereof.

Bioactive agents such as anti-thrombogenic agents, anti-platelet agents, anti-coagulants, statins, vasoactive agents, vasodilator agents, prostaglandins, thrombin inhibitors, plasma proteins, cardiovascular agents, endothelial cells, anti-inflammatory agents, antibiotics, antioxidants, phospholipids, heparins and heparinoids are particularly useful when the object is a stent. Bioactive agents such as analgesics, anti-metabolic agents, antibiotics, growth factors and the like, are particularly useful when the object is an orthopedic implant.

Non-limiting examples of commonly prescribed statins include Atorvastatin, Fluvastatin, Lovastatin, Pravastatin and Simvastatin.

Non-limiting examples of non-steroidal anti-inflammatory drugs include oxicams, such as piroxicam, isoxicam, tenoxicam, sudoxicam, and CP-14,304; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone.

Non-limiting examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, difluorosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

Non-limiting examples of analgesics (pain relievers) include aspirin and other salicylates (such as choline or magnesium salicylate), ibuprofen, ketoprofen, naproxen sodium, and acetaminophen.

Growth factors are hormones which have numerous functions, including regulation of adhesion molecule production, altering cellular proliferation, increasing vascularization, enhancing collagen synthesis, regulating bone metabolism and altering migration of cells into given area. Non-limiting examples of growth factors include insulin-like growth factor-1 (IGF-1), transforming growth factor-β(TGF-β), a bone morphogenic protein (BMP) and the like.

Non-limiting examples of toxins include the cholera toxin, which also serves as an adjuvant.

Non-limiting examples of anti-proliferative agents include an alkylating agent such as a nitrogen mustard, an ethylenimine and a methylmelamine, an alkyl sulfonate, a nitrosourea, and a triazene; an antimetabolite such as a folic acid analog, a pyrimidine analog, and a purine analog; a natural product such as a vinca alkaloid, an epipodophyllotoxin, an antibiotic, a taxane, and a biological response modifier; miscellaneous agents such as a platinum coordination complex, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, or a gonadotropin-releasing hormone analog. Specific examples of chemotherapeutic agents include, for example, a nitrogen mustard, an epipodophyllotoxin, an antibiotic, a platinum coordination complex, bleomycin, doxorubicin, paclitaxel, etoposide, 4-OH cyclophosphamide, and cisplatinum.

As discussed hereinabove, the electropolymerized polymers described herein are preferably designed so as to allow the attachment thereto or incorporation therein of an active substance. The terms “attachment”, “incorporation”, “loading” and any grammatical version thereof are used herein interchangeably to describe in general an interaction between the active substance and the polymer.

Preferably, the interactions by which the active substance is attached to the electropolymerized polymer include any of covalent bonds, non-covalent bonds, biodegradable bonds, non-biodegradable bonds, hydrogen bonds, Van der Waals interactions, hydrophobic interactions, surface interactions, physical interactions and any combination thereof.

The phrase “covalent bonds” is used herein to describe an interaction in which the active substance is covalently bound to the polymer. Covalent bonds are typically formed upon reacting the active substance and the polymer in such conditions that would allow the formation of such a bond.

The covalent bond can be either degradable or non-degradable.

The term “degradable” is used herein interchangeably with the term “biodegradable”, and describes a bond that can be broken down in the body as a result of biological processes, for example, enzymatic processes (by hydrolases, amidases and the like).

The term “non-degradable” is used herein interchangeably with the term “non-biodegradable” and “stable” and describes a bond that is not susceptible to biological processes and hence remains intact for a prolonged time in the body.

“Non-covalent bonds” are used herein to describe interactions that do not involve covalent bonds between the active substance and the polymer, and include, for example, hydrogen bonds, Van der Waals interactions, hydrophobic interactions, physical interactions and surface interactions. Such bonds are typically formed by bringing the reacting substances (e.g., the polymer and the active substance) in a close proximity (e.g., contacting), without particular chemical manipulations, such that the interactions are formed as a result of the nature and characteristics of each of the substances.

Thus, for example, hydrophobic interactions are formed as a result of contacting two hydrophobic reactants. Hydrogen bonds are formed as a result of contacting substances in which at least one has one or more electronegative atom. Surface interactions are formed, for example, when the polymer is porous and enables the entrapment of the active substance within the pores. Physical interactions include surface interactions, as described herein, as well as interactions such as swelling, encapsulation, and the like.

Non-covalent interactions typically result in an electropolymerized polymer in which the active substance can be swelled, absorbed, embedded and/or entrapped.

The attachment of the active substance to the electropolymerized polymer can depend on the nature of the polymer, which, in turn, is determined by the nature of the electropolymerizable monomer used in the electropolymerization process.

The phrase “electropolymerized polymer” is used herein to describe a polymer that can be formed by applying a potential to a solution of its corresponding monomer or monomers. The monomer or monomers are termed “electropolymerizable monomers”.

Representative examples of electropolymerized polymers that are usable in the context of the present embodiments include, without limitation, polypyrroles, polythiophenes, polyfuranyls, poly-p-phenylenes, poly-p-phenylene sulfides, polyanilines, poly(2,5-thienylene)s, fluoroaluminums, fluorogalliums, phtalocyanines, and any combination thereof, whereby the polymers can be used as is or as derivatives thereof in which the backbone unit is substituted by various substances that may provide the surface with the desired characteristics, e.g., polymers, hydrocarbons, carboxylates, amines and the like.

In a preferred embodiment of the present invention, the electropolymerized polymer is formed by electropolymerizing a pyrrole, a thiophene, and derivatives thereof, including oligomers composed of one or more pyrrole residue and one or more thiophene residue. Such oligomers are beneficial since the resulting polymer is characterized by flexibility, stability and high adherence to the metallic surface.

In another preferred embodiment of the present invention, the electropolymerized polymer is formed by electropolymerizing one or more pyrroles, preferably pyrrole derivative(s), one or more thienyls, preferably thienyl derivative(s), and combinations thereof.

As used herein throughout, the term “derivative” with respect to a certain substance or moiety (e.g., pyrrole) describes a substance or moiety that has been subjected to a chemical manipulation, preferably while maintaining its main structural and/or functional characteristics. Such a chemical manipulation preferably includes, for example, substitution, conjugation, and the like.

As discussed hereinabove, the present inventors have now designed and successfully prepared and synthesized a variety of pyrrole and/or thienyl derivatives. These derivatives were designed so provide electropolymerized polymers that enable to attach thereto the active substance via a variety of interactions, depending on the intended use of the article-of-manufacture, the desired release characteristics of the active substance, the desired surface properties of the object and many more.

The preparation and use of various pyrrole and/or thienyl derivatives is exemplified and detailed in the Examples section that follows.

As is demonstrated in the Examples section, it was found that different derivatives of pyrrole and/or thienyl result in different characteristics of the formed electropolymerized polymer, in terms of mechanical properties, chemical properties and in terms of the efficiency to embed therein active substances.

Thus, it was found, for example, that electropolymerization of N-alkyl derivatives of pyrrole forms a thin, uniform and porous coating that surprisingly adhere well to metal surfaces, particularly stainless steel. The thickness of the coating is well controlled by the number of cycles applied. For example, a mixture of N-pyrrole propanoic acid, N-pyrrole propanoic acid butyl ester and hexyl ester form a flexible thin porous coating onto a coronary stent that do not tear even upon 50% expansion. Coatings of 0.1 to 2 micron thick were achieved by applying 1 to 20 electrocycles, respectively. Furthermore, these N-alkyl polypyrroles porous coatings absorb a large amount of a drug (paclitaxel, estradiol, serolimun, dexamethasone) by immersion of the coated element in an organic solution of the drug and solvent evaporation. Such a loaded coating releases the absorbed drug during a period of a few weeks with little burst effect.

Additional pyrrole and/or thienyl derivatives have been further found beneficial for use as monomers for deposing electropolymerized polymer on conductive surfaces and attaching thereto various active substances.

Thus, for example, electropolymerizable monomers which comprise two or more (e.g., 3, 4 and up to 6) electropolymerizable moieties being linked to one another, were designed, as is described in detail hereinunder. Depending on the selected moieties and the presence and nature of the moiety (spacer) linking therebetween, chemical and mechanical (e.g., flexibility) properties of the resulting polymer can be achieved.

By manipulating the nature of the electropolymerized polymer, attachment of an additional polymer can be performed, such that according to an embodiment of the present invention, the article-of-manufacture further comprises at least one additional polymer attached to the electropolymerized polymer.

The additional polymer can be, for example, an additional electropolymerized polymer and/or a chemically-polymerized polymer.

The additional polymer is preferably a hydrophobic polymer, a biodegradable polymer, a non-degradable polymer, a hemocompatible polymer, a biocompatible polymer, a polymer in which the active substance is soluble, and/or a flexible polymer, and can be selected so as to affect (i) mechanical, physical and/or chemical characteristics of the coating (e.g., charge, wettability, flexibility, stability and the like); and/or (ii) the release profile of an active substance.

In one embodiment, the additional polymer is an electropolymerized polymer. Thus, for example, a multi-layered polymeric coating can be achieved by repeatedly performing an electropolymerization process, using the same or different monomers each time.

In another embodiment, the additional polymer is a chemically-polymerized polymer. Such a polymer can be attached to the electropolymerized polymer by non-covalent interactions and thus can be swelled, absorbed or embedded within said electropolymerized monomer. Alternatively, the polymer can be covalently attached to the electropolymerized monomer.

Further alternatively, the additional polymer forms a part of said electropolymerized polymer. As is exemplified in the Examples section that follows, electropolymerizable monomers can be designed so as to have a chemically-polymerizable group attached thereto, such that upon electropolymerization, the chemically-polymerizable group can participate in the formation of a chemically-polymerized polymer. Thus, the formed chemically-polymerized polymer forms a part of the electropolymerized polymer.

In another alternative, the additional polymer is formed by chemically polymerizing corresponding monomers onto the electropolymerized polymer. The thus formed polymer can form an interpenetrating system with the electropolymerized polymer, via, for example, cross-linking, and thus forms a part of the electropolymerized polymer.

In yet another alternative, the electropolymerizable monomer can be designed to include a reactive group that can participate in the chemical polymerization of the additional polymer. Such a reactive group can be, for example, a photoactivatable group, which can initiate polymerization upon irradiation, or a polymerization-initiating group, which can initiate a polymerization process in the presence of a catalyst. Examples of the latter include, but are not limited to vinyl group, allyl groups and the like.

In each of these alternatives, a multi-layered coating is obtained. Such a multi-layered coating can be used for controlling the relapse characteristics of the active substance. The active substance can be attached either to the electropolymerized monomer and/or to the additional polymer, as is exemplified hereinbelow, or, alternatively, be entrapped therebetween.

Thus, for example, the active substance can be attached (either covalently or non-covalently) to the electropolymerized polymer, which is further coated by an additional polymer. Optionally, the active substance can be attached (either covalently or non-covalently) to the additional polymer, whereby the latter is embedded within the electropolymerized polymer, and thus, the active substance is attached to the electropolymerized polymer via the additional polymer.

A multi-layered polymeric coating can therefore be achieved by repeatedly performing an electropolymerization process, using the same or different monomers each time.

Alternatively, a multi-layered coating can be achieved by interacting the electropolymerized polymer with an additional polymer, such that the latter is embedded in the electropolymerized polymer due to hydrophobic interactions.

Further alternatively, a multi-layered coating can be achieved by covalently attaching a chemically-prepared polymer to the electropolymerized polymer. This can be achieved either by utilizing, in the electropolymerization process, monomers that are substituted by a polymer, or by utilizing monomers that have a polymerizable group, which may react to form the chemically-polymerized polymer concomitant with or subsequent to the formation of the electropolymerized polymer. Thus formed additional polymers eventually form a part of the electropolymerized polymer.

Further alternatively, the chemically-polymerized polymer can be formed by utilizing electropolymerizable monomers that have a reactive group, which is capable of participating in the formation of a chemically-polymerized polymer. Such a reactive group can be for example, a photoactivatable group. Thus, the formed electropolymerized polymers have such photoactivatable groups, which upon irradiation, may react with various monomers and activate the polymerization thereof on the electropolymerized monomer. Such a reactive group can also be, for example, a polymerization-initiating group. Thus, the formed electropolymerized polymers have such groups, which when contacted with various monomers, initiate the polymerization thereof such that a cross-linked, interpenetrating system is formed.

The additional polymer, or the monomers used for its preparation, are selected so as to provide either degradable or non-degradable bonds.

Suitable non-degradable polymers for use in the context of the present embodiments are those that are hemo- and biocompatible, non-rigid (so as to allow their expansion when applied on expandable stents) and/or are soluble in common organic solvents (e.g., chlorinated hydrocarbons, cyclohexane, ethyl acetate, butyl acetate, N-methylpyrrolidone, and lactate esters), so as to enable their loading onto a coated surface. Representative examples include polyurethanes that are commonly used in medical devices, silicone, polyacrylates and methacrylates, particularly the copolymers of lauryl methacrylates. Polymers containing butadiene and isoprene are also suitable.

Suitable biodegradable polymers for use in the context of the present embodiments include, without limitation, polymers that are based on lactic acid, glycolic acid and caprolactone. These polymers can be applied onto and into the electropolymerized coating by dipping the coated surface in a diluted solution of the polymer or of the polymer with a bioactive agent and other additives that are used to facilitate and/or control the loading and release of the bioactive agent. Of particular interest are the homopolymers of lactic acid, copolymers of lactic acid with glycolic acid and copolymers containing caprolactone.

When attached to the electropolymerized polymer, the polymers can be loaded by dipping or spraying a dilute solution of the polymer such that the polymer is well and uniformly distributed within and/or onto the electropolymerized polymer. To increase the loading of the polymer, several serial dipping can be applied. The dipping or spraying of the polymer solution can be carried out under various temperature and environmental conditions that provide a uniform coating without any access of the polymer at certain parts of the implant.

By manipulating the nature of the polymer and the electropolymerizable monomer utilized and the conditions and stage at which the active substance is loaded, the release profile of the active substance can be controlled.

Thus, for example, the polymer solution may contain bioactive agents dissolved or dispersed in the polymer solution, or particles loaded with the bioactive agent. Different dip or spray coatings are applied.

In one example, a porous polypyrrole coating, obtained as described above, is loaded with a bioactive agent prior to applying a non-degradable polymer thereon, such that the loaded electropolymerized polymer is sealed with a thin layer of a non-degradable polymer to better control the release of bioactive agent from the coating and/or improve the hemo- and biocompatibility as well as the adherence, attachment and stability of the coating onto the device.

In another example, the chemical polymerization solution can contain the bioactive agent in an amount as high as 50% of the polymer content, such that when applied onto the electropolymerized polymer, a polymeric matrix of the chemical polymer and the electropolymerized polymer is formed, which is loaded by the bioactive agent and enables its release during an extended time period. To further control the release rate of the bioactive agent, an additional polymer can be applied onto the previously loaded polymer-bioactive agent mixture. Such a technique results in a high load of the active substance (as high as 50 weight percents) within the polymeric matrix.

Alternatively, the electropolymerized polymer can be contacted with chemically-polymerizable monomers that upon initiation polymerize to form an interpenetration network with the electropolymerized polymer. When such chemically-polymerizable monomers are added into the electropolymerization solution, an electropolymerized polymeric coating in which these monomers are entrapped can be formed.

The polymerization of the monomers entrapped within the coating can be performed by initiation with a radical source such as benzoyl peroxide that initiate the polymerization by either heat or light that split benzoyl peroxide into radicals. Alternatively, the monomers are loaded into electropolymerized polymer without an initiator and the polymerization occurs when immersing the monomer-loaded coating into an aqueous solution containing a redox radical system that initiates polymerization at the water-coating interface. The amount of the interpenetrating polymer is controlled by the monomer concentration in the solution, the solvent used and the polymerization process. The properties of the coating are controlled by the monomer composition, the loading in the electropolymerized matrix, and the degree of crosslinking. For example, including hydroxyl ethyl methacrylate (HEMA) or polyethylenglycol acrylate (PEG-acrylate) at an increasing amount in the monomer composition, increases the hydrophilicity of the coating and even provide a slippery and smooth coating when immersed in water. On the other hand, a hydrophobic nature of the coating may be obtained when the amount of lauryl methacrylate (LMA) or other alkyl acrylates in the polymer composition is increased. Increasing the amount diacrylates or methacrylates, increases the rigidity and stiffness of the coating. Crosslinking agents can be ethylene glycol dimethacrylate, PEG-diacrylate, ethylenebis-acrylamide, divinyl benzene and other crosslinkers commonly used in biopolymerization of acrylates.

Electropolymerized polymers that have amine or hydroxyl groups can be further used for forming biodegradable polymers that are based on lactide, glycolide or caprolactone by ring opening polymerizations of these lactones, in which the hydroxyl or amine serve as polymerization-initiating group.

For better entrapment of the drug, so as to achieve a prolonged release period, a hydrophobic polymer is preferred. However, for better compatibility with tissue, a hydrophilic surface is preferred. Thus, manipulations can be made such that the outer coating is a hydrophilic polymer coating, which is applied onto a hydrophobic electropolymerized polymer loaded with the active agent.

Covalent attachment of active substances to the electropolymerized polymer is widely described in the Examples section that follows, and in WO 01/39813 and U.S. patent application Ser. No. 10/148,665.

For covalently attaching bioactive agents, electropolymerizable monomers that include the bioactive agent covalently attached thereto can be used. Particularly useful monomers for that purpose include N-alkyl pyrrole derivatives possessing functional groups such as carboxylic acid and derivatives thereof (e.g., acyl halide, ester), amine, hydroxyl, vinyl, acetylene and thiol. These groups can be used for binding small and large molecules onto the coating such as PEG chains, fatty acid chains, polymer chains, and fluorescent markers.

Of particular interest are the binding of fatty acids, alcohols and polymers via amidation or esterification of carboxylic acids, such that one of the active substance and the electropolymerized polymer includes hydroxyl or amine whereby the other include a carboxylic acid or a derivative thereof.

The methodologies described above are exemplified in the Examples section that follows. As is demonstrated therein, coatings having a thickness in the ranges of from about 0.1 micron to 10 microns, and preferably from 0.1 micron to 5 microns were obtained. The controlled release of bioactive agents from exemplary coatings has also been demonstrated.

For implementing these methodologies and thus control the properties of the coating and the release profile of an active substance that is embedded therein, novel electropolymerizable monomers, having special characteristics, have been designed.

Thus, according to another aspect of the present invention, there is provided an electropolymerizable monomer which has one or more of the following functional groups:

(i) a functional group capable of enhancing an adhesion of an electropolymerized polymer formed from the electropolymerizable monomer to a conductive surface;

(ii) a functional group capable of enhancing absorption, swelling or embedding of an active substance within an electropolymerized polymer formed from the electropolymerizable monomer;

(iii) a functional group capable of forming a chemically-polymerized polymer;

(iv) a functional group capable of participating in the formation of a chemically-polymerized polymer;

(v) a functional group capable of providing an electropolymerized polymer formed from the electropolymerizable monomer having a thickness that ranges from about 0.1 micron to about 10 microns;

(vi) a functional group capable of enhancing the flexibility of an electropolymerized polymer formed from the electropolymerizable monomer; and

(vii) a functional group capable of covalently attaching an active substance thereto.

Also included are functional groups that can serve as a nucleation center for growing crystals onto the surface of the device.

Thus, for example, the presence of a functional group such as ω-carboxyalkyl group, wherein the alkyl preferably has at least 3 carbon atoms, in an electropolymerizable monomer provides for enhanced adhesion of an electropolymerized polymer formed from the electropolymerizable monomer to a conductive surface, enhanced absorption, swelling or embedding of an active substance within an electropolymerized polymer formed from the electropolymerizable monomer, enables to covalently attach an active substance thereto and/or provides an electropolymerized polymer having a thickness that ranges from about 0.1 micron to about 10 microns.

Electropolymerizable monomers that are substituted and/or are interpreted by a polyalkylene glycol residues provide for enhance flexibility and uniformity of the coating.

Additional examples of functional groups that are capable of enhancing the flexibility of the formed electropolymerized polymer include, without limitation, alkylene glycols, non-linear alkylene chains, and residues containing urethane (—NH—C(═O)O—), carboxy (—C(═O)—O—) and sulfide bonds (—S—S—).

Additional examples of functional groups that are capable of enhancing absorption, swelling or embedding of an active substance within the formed electropolymerized polymer include, without limitation, amine, carboxylate, hydroxamic acid, sulfonate, sulfate, epoxide, thiol and vinyl.

Functional groups that are capable of forming a chemically-polymerized polymer include, for example, an allyl group and a vinyl group, as is detailed herein and is further exemplified in the Examples section that follows.

Functional groups that are capable of participating in the formation of a chemically-polymerized polymer include, for example, photoactivatable group and polymerization-initiating groups, as is detailed herein and is further exemplified in the Examples section that follows.

Thus, according to further aspects of the present invention there are provided novel electropolymerizable monomers.

In one embodiment, there is provided an electropolymerizable monomer which comprises at least two electropolymerizable moieties being linked to one another.

As used herein, the phrase “electropolymerized moiety” describes a residue of an electropolymerizable monomer. As is well-known in the art, the term “residue” describes a major portion of a molecule which is linked to another chemical moiety (e.g., another electropolymerizable moiety or a spacer).

The two or more electropolymerizable moieties can be the same or different, and can be selected from, for example, pyrrole, thienyl, furanyl, thiophene, each being optionally substituted at one or more position thereof. When substituted, the electropolymerizable moiety can include, for example, one or more substituents such as alkyl, alkenyl, alkynyl, polyalkylene glycol, cycloalkyl, each being optionally further substituted by one or more groups such as aryl, halo, amine, hydroxy, thiohydroxy, carboxy (C(═O)OR, where R is hydrogen, halo, alkyl and the like).

The electropolymerizable moieties in such electropolymerizable monomers can be linked to one another directly, via a covalent bond, or indirectly, via a spacer. When more than two electropolymerizable moieties are present in the monomer, a combination of the above can be effected such that, for example, two moieties are linked directly to one another and two moieties are linked via a spacer.

The spacer preferably comprises a substituted or unsubstituted, saturated or unsaturated hydrocarbon chain, optionally interrupted by one or more heteroatoms (e.g., O, N or S). Examples include, without limitation, a substituted or unsubstituted alkyl, a substituted or unsubstituted alkenyl, a substituted or unsubstituted alkynyl, a substituted or unsubstituted cycloalkyl, and a substituted or unsubstituted polyalkylene glycol. When substituted, the one or more substituents can be, for example, halo, alkyl, amine, hydroxy, carboxy. The hydrocarbon chain can be attached to each of the electropolymerizable moieties either directly (e.g., a sigma bond) or via a bonding member such as an amide bond, an ester bond, an ether bond and the like.

Thus, exemplary electropolymerizable monomers according to this embodiment, comprise two pyrrole moieties linked to one another via a PEG chain. Such monomers are referred to herein interchangeably as bis-pyrrole PEG and PEG dipyrrole. The PEG chain preferably has a molecular weight in the range of from about 100 Da to about 600 Da.

Additional exemplary electropolymerizable monomers according to this embodiment comprise one or more pyrrole moieties and one or more thienyl moieties attached to one another directly or via a short space (e.g., ethane, ethene, etc.).

All of the above described moieties, groups, and substituents can be further substituted as described herein.

Representative examples of the electropolymerizable monomers according to this embodiment include, without limitation, 1,2,6-tri(N-propanoyl pyrrole)-hexane, 1,1′,1″,1′″-tetra(N-propanoyl pyrrole)-methane, bis-pyrrole-PEG, 1,1′-di(2-thienyl)ethylene, 3-dimethylamino-1-(2-thienyl)-propanone, 1,4-di(2-thienyl)-1,4-butandiol, and 1,2-di(2-pyrrolyl)-ethene.

In another embodiment, the electropolymerizable monomer comprises at least one electropolymerizable moiety, as described herein, and at least one functional group that is capable of forming a chemically-polymerized polymer being attached to the electropolymerizable moiety or moieties.

As used herein throughout the phrase “functional group capable of forming a chemically-polymerized polymer” describes a polymerizable group, which when subjected to the appropriate chemical conditions can be polymerized. Appropriate chemical conditions include, for example, catalytic initiation of radical chain polymerization, photo-initiation of radical chain polymerization, catalytic initiation of ring opening polymerization, cross-linking (presence of a cross-linking agent), and co-polymerization (presence of a co-polymer, and optionally a polymerization catalyst or cross-linking agent).

Exemplary such functional groups include, without limitation, vinyl and allyl groups, which upon catalytic initiation can form a polyalkane or polyalkene, acrylic acid or acrylamide, lactones, which can by subjected to ring opening polymerization, phosphates, which can be cross-linked in the presence of divalent metal atoms, and the like.

In another embodiment, the electropolymerizable monomer comprises at least one electropolymerizable moiety, as described herein, and at least one functional group that is capable of participating in the formation of a chemically-polymerized polymer being attached to the electropolymerizable moiety or moieties.

As used herein throughout, the phrase “functional group capable of participating in the formation of a chemically-polymerized polymer” describes a group that can catalyze or induce polymerization of chemically-polymerizable monomers. Such a functional group can be, for example, a photoactivatable group, which upon irradiation, becomes a reactive group that capable of initiating a polymerization process such as, for example, radical chain polymerization or ring opening polymerization, as described herein. Alternatively, the functional group can be a cross-linking group, which can act as a cross-linking agent.

Representative examples of photoactivatable groups include, without limitation, benzophenone derivatives.

Representative examples of cross-linking groups include, without limitation, acrylate, acrylamide, and divinyl benzene.

In each of the embodiments described herein the functional group can be attached directly to the electropolymerizable moiety (e.g., via a sigma bond) or indirectly via a bonding member such as an amide bond, an ester bond, an ether bond and the like. Representative examples of electropolymerizable monomers according to these and other embodiments of the present invention are presented in the Examples section that follows.

The electropolymerizable monomers described herein, as well as the methodologies described in detail hereinabove, have been beneficially utilized for obtaining the articles-of-manufacture described herein.

Based on the methodologies described hereinabove, there is provided, according to another aspect of the present invention, a process of preparing the article-of-manufactures described herein. The process is effected by: providing an object having a conductive surface; providing a first electropolymerizable monomer; providing an active substance; electropolymerizing the electropolymerizable monomer, to thereby obtain an object having the electropolymerized polymer attached to at least a portion of a surface thereof; and attaching the active substance to the electropolymerized polymer.

Attaching the active substance to the electropolymerized polymer is effected via any of the interactions described hereinabove.

In one embodiment of this aspect of the present invention, the active substance is swelled, absorbed, embedded and/or entrapped within the electropolymerized polymer.

Attaching the active substance according to this embodiment can be performed by: providing a solution containing the active substance; and contacting the object having the electropolymerized polymer attached to its surface with the solution.

In another embodiment, the article-of-manufacture further comprises an additional polymer attached to the electropolymerized polymer, and the process further comprises: attaching the additional polymer to the electropolymerized polymer, to thereby provide an object having an electropolymerized polymer onto at least a portion of a surface thereof and an additional polymer attached to the electropolymerized polymer.

In another embodiment, the additional polymer is an electropolymerized polymer and the process is further effected by providing a second electropolymerizable monomer; and electropolymerizing the second electropolymerizable monomer onto the object having the electropolymerized polymer onto at least a portion of a surface thereof.

The electropolymerizing of the second monomer can be performed prior to, concomitant with and/or subsequent to attaching the active substance.

In yet another embodiment, the additional polymer is a chemically-polymerized polymer that is swelled, absorbed or embedded within the electropolymerized monomer, and the process is further effected by providing a solution containing the chemically-polymerized polymer; and contacting the object having said electropolymerized polymer attached to said surface with said solution.

The contacting can be performed prior to, concomitant with and/or subsequent to attaching said active substance.

Alternatively, the process is effected by providing a solution containing a monomer of the chemically-polymerized polymer; and polymerizing the monomer while contacting the object having the electropolymerized polymer attached to the surface with the solution.

The polymerization can be performed prior to, concomitant with and/or subsequent to attaching the active substance.

In still another embodiment, the additional polymer is a chemically-polymerized polymer that forms a part of the electropolymerized polymer and providing the first electropolymerizable monomer comprises providing an electropolymerizable monomer that has a functional group that is capable of interacting with or forming the additional polymer.

In cases where the functional group is selected capable of forming the additional polymer, the process further comprises subjecting the object having the electropolymerized polymer attached thereto to a chemical polymerization of the functional group.

The chemical polymerization can be performed prior to, concomitant with and/or subsequent to attaching the active substance.

In cases where the functional group is selected capable of participating is the formation of the additional polymer, the process further comprises: providing a solution containing a substance capable of forming the additional polymer; and contacting the object having the electropolymerized polymer attached to the surface with the solution.

The contacting can be performed prior to, concomitant with and/or subsequent to attaching said active substance.

The functional group in this case can be, for example, a photoactivatable group, a cross-linking group and/or a polymerization-initiating group, as described in detail hereinabove.

In an additional embodiment, the active substance is covalently attached to the electropolymerized polymer, the electropolymerizable monomer has the active substance covalently attached thereto and attaching the active substance to the electropolymerized polymer is effected by electropolymerizing the monomer.

Alternatively, the first electropolymerizable monomer has a reactive group capable of covalently attaching the active substance and attaching the active substance is effected by reacting a solution containing the active substance with the object having the electropolymerized polymer attached to at least a portion of a surface thereof.

The present inventors have further designed novel methods for pre-treating a conductive surface prior to the formation of the electropolymerized polymer, so as to enhance the adhesion of the electropolymerized polymer to the surface. The process described herein can therefore further include such a pre-treatment of the surface. These pre-treatment methods according to the present invention are effected by subjecting the surface to one or more of the following procedures:

manually polishing the surface, preferably using a grit paper; and rinsing the surface with an organic solvent;

contacting the surface with an acid such as, for example nitric acid, sulfonic acid or any other inorganic or organic acid; rinsing the surface with an aqueous solvent; and subjecting the surface to sonication; and

subjecting the surface to sonication; and rinsing the surface with an organic solvent, an aqueous solvent or a combination thereof. Preferably, the sonication is performed in the presence of carborundum and/or in an organic solvent.

Representative examples of preferred methods for treating a surface prior to electropolymerizing thereon according to the present embodiments are widely described in the Examples section that follows.

The present invention therefore provides various articles-of-manufacture that can be prepared by controlled, yet versatile, processes, resulting in objects coated by various beneficial active substances, whereby the coatings are characterized by enhanced adherence, enhanced density of the active substance and improved surface characteristics, as compared with the presently known coatings. The processes described herein enable to finely control various characteristics of the coating, including, for example, its hydrophobicity/hydrophilicity, its flexibility, the release rate of the active substance, the amount of the loaded active substance, and more, as is detailed herein.

When the articles-of-manufacture described herein are coated implantable devices, these articles-of-manufacture can be beneficially used in the treatment of conditions in which implanting a medical device, and particularly such a device loaded with bioactive agents, is beneficial.

Such conditions include, for example, cardiovascular diseases such as, but not limited to, atherosclerosis, thrombosis, stenosis, restenosis, and in-tent stenosis, cardiologic diseases, peripheral vascular diseases, orthopedic conditions, proliferative diseases, infectious diseases, transplantation-related diseases, degenerative diseases, cerebrovascular diseases, gastrointestinal diseases, hepatic diseases, neurological diseases, autoimmune diseases, and implant-related diseases.

The active substance attached to the device is selected suitable for treating the disease.

The present inventors have further designed a device, cartridge and system, which enable an efficient preparation of various medical devices that are coated and loaded by active substances using the methodologies described herein.

Thus, according to an additional aspect of the present invention, there is provided a device for holding a medical device while being subjected to electropolymerization onto a surface thereof, which comprises a perforated encapsulation, adapted to receive the medical device, and at least two cups adapted for enabling electrode structures to engage with said perforated encapsulation hence to generate an electric field within the perforated encapsulation.

The perforated encapsulation is preferably further designed and constructed to allow fluids and chemicals to flow therethrough.

According to another aspect of the present invention, there is provided a cartridge, comprising a plurality of the holding devices described above, and a cartridge body adapted for enabling the plurality of holding devices to be mounted onto said cartridge body. Preferably, the cartridge comprises more than 3 holding devices.

According to another aspect of the present invention, there is provided a system for coating medical devices, which comprises, in operative arrangement, at least one holding device as described above, a conveyer and a plurality of treating baths arranged along the conveyer, wherein the conveyer is designed and constructed to convey the holding device such that the holding device is placed within each of the treating baths for a predetermined time period and in a predetermined order.

The system preferably further comprises a cartridge having a cartridge body adapted for enabling the holding device to be mounted onto the cartridge body.

The plurality of treating baths in the system include, for example, one or more of a pretreatment bath, a washing bath, an electrochemical polymerization bath, a rinsing bath, a chemical polymerization bath and an active substance solution bath, depending on the coating and loading methodology used. Preferably, at least two of the baths are an electrochemical polymerization bath and an active substance solution bath.

The electrochemical polymerization bath preferably comprises at least one of electrode structure, mounted on a base of the electrochemical polymerization bath and connected to an external power source.

Further preferably, the conveyer is operable to mount the at least one holding device on the at least one electrode structure, thereby to engage the at least one electrode structure with a first side of the perforated encapsulation.

The system preferably further comprises an arm carrying at least one electrode structure and operable to engage the electrode structure with a second side of the perforated encapsulation.

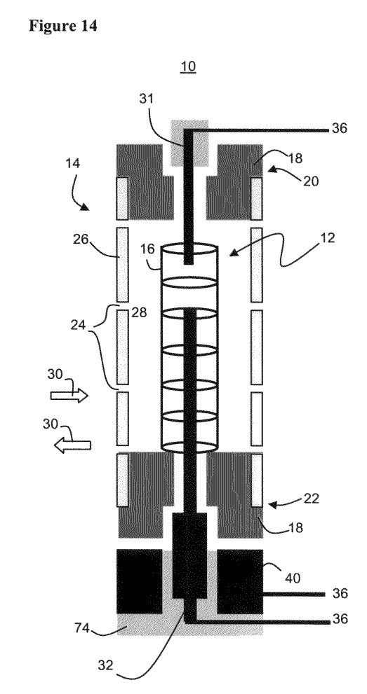

Referring now to the drawings, FIG. 14 illustrates a device 10 for holding a medical device 12 while being coated, according to a preferred embodiment of the present invention. Medical 12 is preferably a stent, as is illustrated in this figure. Holding device 10 comprises a perforated encapsulation 14 which receives medical device 12. Assembly 12 is shown in FIG. 14 as an expandable tubular supporting element 16 which can be used, for example, when the medical device is a stent assembly. Preferably, but not obligatorily, encapsulation 14 has a tubular (e.g., cylindrical shape). Device 10 preferably holds medical device 12 throughout the entire treatment of assembly 12. Thus, device 10 can hold assembly 12 while being treated in, for example, a chemical treatment bath, an electrochemical treatment bath, an ultrasonic bath, a drying zone, a drug loading bath and the like.

Perforated encapsulation 14 comprises a plurality of holes 24 formed on its wall 26 so as to allow various chemicals solutions 30 to flow from the respective treatment bath, through wall 26 and into an inner volume 28 of encapsulation 14 thereby to interact with medical device 12 and/or supporting element 16. Additionally, holes 24 preferably allow chemicals solutions to flow out of inner volume 28, for example when device 10 is pulled out of the respective treatment bath.

Device 10 further comprises two or more cups 18 covering a first end 20 and a second end 22 of encapsulation 14. Cup 18 can be made of, e.g., stainless steel. According to a preferred embodiment of the present invention cups 18 are adapted for enabling various electrode structures, designated in FIG. 1 by numerals 31 and 32, to engage with encapsulation 14. This embodiment is particularly useful when assembly 12 is subjected to electrochemical polymerization. Thus, a reference electrode can be inserted from one side and a counter electrode can be inserted from the opposite side. Additionally, a working electrode can be positioned near, say, a few millimeters apart from cup 18 such that, when the electrodes are connected to a power source (not shown), for example, via communication lines 36, an electric field is generated and redox reaction is driven on a working electrode 40. A polymerization process is thus initiated within volume 28 and member 16 is coated by the polymer film.

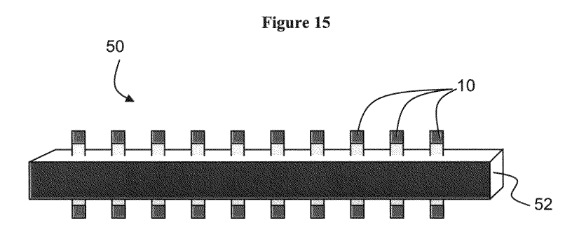

Several, preferably three or more holding devices can be employed for coating several medical devices simultaneously. FIG. 15 is a schematic illustration of a cartridge 50 of holding devices. The principles and operations of each of the holding devices on cartridge 50 is similar to the principles and operations of device 10 as further detailed hereinabove. Cartridge 50 serves for placing several holding devices together in the treatment baths. In the exemplified configuration of FIG. 15 cartridge 50 holds 10 devices, but this need not necessarily be the case, and any number of holding devices can be mounted on a body 52 of cartridge 50. The body of the cartridge 50 is preferably designed to be mounted on a conveyer that places cartridge 50 in the treatment bathes as further detailed hereinbelow.

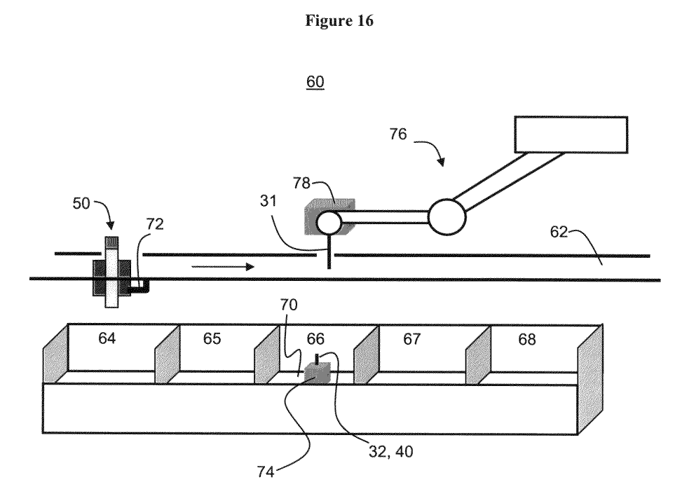

Reference is now made to FIG. 16 which is a schematic illustration of a system 60 for coating one or more medical devices, according to a preferred embodiment of the present invention. System 60 preferably comprises, in operative arrangement, one or more holding devices (e.g., device 10). When several holding devices are used, the devices are preferably mounted on a cartridge, for example, cartridge 50.

System 60 further comprises a conveyer 62 and a plurality of treating baths arranged along conveyer 62. In the representative example shown in FIG. 16, system 60 comprises five treating baths designated 64, 65, 66, 67 and 68. Thus, for example, bath 64 can be used as a pretreatment bath in which the medical device is subjected to chemical and mechanical treatments so as to prepare the medical device to a uniform and adherent coating. Bath 65 can be used for washing, bath 66 can be used for electrochemical polymerization, bath 67 can be used for cleaning and bath 68 can be an active substance solution bath, e.g., for drug loading. Other baths or treatment zones are also contemplated.

Conveyer 62 conveys the holding device(s) such that the device is placed within each treating baths in a predetermined order. Thus, for example, in the exemplified embodiment of FIG. 16, conveyer 62 places the device first in bath 64, then in bath 65 etc. Additionally, conveyer 62 controls the time period at which the device spends in each bath. This can be achieved by designing conveyer 62 to pull the device from the respective bath after a predetermined time period and place it in the next bath in line. Conveyer 62 is preferably manufactured with a lever 72 or any other mechanism for placing the device in the baths before treatment and pulling it out thereafter.

According to a preferred embodiment of the present invention the electrochemical polymerization bath comprises electrode structures (e.g., counter electrode 32 and working electrode 40) mounted on base 70 thus forming a lower electrochemical polymerization unit. The electrode structures preferably protrude out of an isolating material 74 (see also FIG. 14) and connected to a power source (not shown). In operation, conveyer 62 mounts the holding device on the electrode structure(s), which in turn engage with the one side of the device. System 60 can also comprise an arm 76 carrying one or more electrode structure (e.g., reference electrode structure 31), which preferably protrudes out of an isolating material 78. Arm 76 and electrode 31 thus form an upper electrochemical polymerization unit.

Once the holding device is mounted on electrodes 32 and/or 40, arm 76 causes electrode 31 to engage with the other (upper in the present embodiment) side of the holding device. Being in electrical communication with the electrodes, the medical device in the holding device can be subjected to the electrochemical polymerization as known in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples which, together with the above descriptions, illustrate the invention in a non limiting fashion.

### MATERIALS AND INSTRUMENTAL METHODS

Chemicals were generally purchased from known vendors such as Sigma, Fluka, Aldrich and Merck and were used without further purification, unless otherwise indicated.

316L Stainless steel plates were purchased from Mashaf Co. (Jerusalem, Israel)

316L Stainless steel Stents, 12 mm long, inflatable to 3 mm diameter were, purchased from STI, Cesaria, Israel. All aqueous solutions were prepared from deionized water (Mili-Q, Milipore).

NMR measurements: ¹H-NMR, ¹³C-NMR, ¹⁹F-NMR, and ³¹P-NMR spectra were obtained on Bruker AC-200, DPX-300 and DMX600 spectrometers. For CDCl₃ and acetone-d₆ solutions, chemical shifts are expressed in ppm downfield from Me₄Si used as internal standard. For D₂O solutions the HOD peak was taken as δ=4.79 (¹H-NMR spectra) or the peak of a small amount of added MeOH taken as δ=49.50 (¹³C-NMR spectra).

MS measurements: Mass spectra were obtained on a MALDI spectrometer (CI=chemical ionization, DCI=desorption chemical ionization, EI=electron ionization).

SEM measurements: The surface morphologies of the coated electrodes were measured by high resolution scanning electron microscopy (HR SEM) using a sirion scanning microscope (FEI Company, Holland) equipped with shottky type field emission source at 10 kV accelerating voltage. Samples were gold coated before subjected to analysis.

HPLC analyses: high-performance liquid chromatography was performed using Hewlett Packard (Waldbronn, Germany) system composed of an HP 1100 pump, HP 1050 UV detector, and HP ChemStation data analysis program using a C18 reverse-phase column (LichroCart^{R} 250-4, Lichrospher^{R} 100, 5 μm). All measurements were carried out at 230 nm.

### Example 1

### Preparation of Pyrrole Derivatives

The following describes the preparation of a variety of electropolymerizable pyrrole monomers, derivatized by functional groups, which are suitable for use in the context of the present invention.

Preparation of Carboxylic Acid- or Amino-Containing Pyrrole Derivatives—General Procedure:

The preparation of carboxylic acid or amino containing pyrrole analogues was conducted based on known protocols by Yon-Hin et al, [Anal. Chem. 1993, 65, 2067-2071], unless otherwise indicated.

Preparation of N-(3-aminopropyl)-pyrrole (APP)—Route A: N-(2-cyanoethyl)pyrrole was reduced with LiAlH₄ in dry diethyl ether, using the general procedure described above, using N-(2-cyanoethyl)pyrrole (available from Aldrich Chemicals) as starting material. N-(3-aminopropyl)-pyrrole was synthesized by reduction of N-(2-cyanoethyl)pyrrole with LiAlH4 in dry diethyl ether in a 90% yield and was identified by H-NMR and IR (data not shown).

Preparation of N-(3-aminopropyl)-pyrrole (APP)—Route B: In an alternative synthetic route, APP Was prepared as depicted in Scheme 1 below.

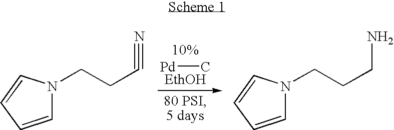

To 2-cyanoethyl pyrrole (10 grams, 83.3 mmol) dissolved in 50 ml methanol, 1 gram of 10% Pd—C were added and the vessel was connected to the hydrogenation system under 70 PSI for 4 days. The solids were precipitated off, the filtrate was collected and the volatiles were removed under reduced pressures. The obtained amine was purified on silica gel chromatography using 20-50% methanol in CHCl₃ as eluent, to afford N-(3-aminopropyl)-pyrrole in a 90% yield. The brownish viscous oil was characterized using NMR (data not shown) and ESI-MS.

ES-MS: m/z=122, 126, 153, 132, 339.

Preparation of N-(2-carboxyethyl)pyrrole (PPA): As depicted in scheme 2, N-(2-cyanoethyl)pyrrole was hydrolyzed in aqueous KOH, according to general procedure mentioned above.

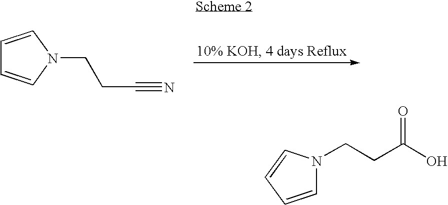

N-(2-Cyanoethy) pyrrole (10 ml, 83.23 mmol) was refluxed in a mixture of 20 grams KOH solution in 50 ml DDW and 10 ml ethanol for 4 days. Once the ammonia evolvement was ceased, the reaction mixture was allowed to cool to room temperature and the solution was acidified using concentrated hydrochloric acid until pH of about 4-5 was reached. The acid was extracted from the reaction mixture with 4×100 ml fractions of CH₂Cl₂. After drying on anhydrous sodium sulfate the organic solvents were removed to dryness under reduced pressure. The yellowish gum product N-(2-carboxyethyl)pyrrole, solidified after cooling and was obtained in a yield of 80% (melting point 58-59° C.).

¹H-NMR (DMSO-d₆): δ=6.749-6.735 (d, 1H), 5.964-5.948 (d, 1H), 4.103-4.058 (t, 2H, CH₂—), 2.661-2.614 (t, 2H, CH₂—) ppm.

MS (ES-MS): m/z (%)=164.6 (MW+Na+H⁺).

Preparation of N-(2-Carboxyethyl)pyrrole-NHS(PPA-NHS):

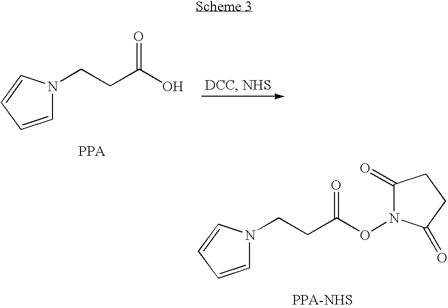

As depicted in scheme 3 above, 2-Carboxyethypyrrole (5 grams, 36 mmol) was dissolved in 70 ml ethyl acetate under calcium chloride tube. To the stirred solution 1.1 equivalent of dicyclohexyl carbodiimide (DCC) and N-hydroxysuccinamide (NHS) were added and stirred continuously. After a while, a white precipitate of DCU was formed. The mixture left to stand at room temperature for overnight, and the precipitate was filtered off and washed with two fractions of 50 ml ethyl acetate. The ethyl acetate fractions were collected and the solvents were removed under reduced pressure until dryness. The white colored residue was collected and stored at −5° C. until use. The product was identified by ¹H-NMR (data not shown).

Preparation of PPA-O-PEG-OH:

As depicted in scheme 4, pyrrolylation of HO-PEG-OH was established through an esterification process in toluene using azeotropical reflux with p-toluene sulfonic acid (PTSA) catalysis.

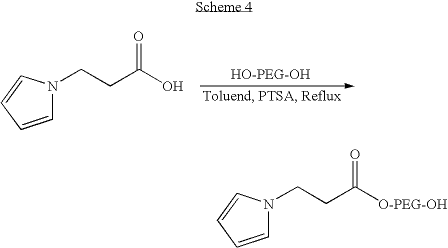

Using the procedure described above, equimolar amounts of PPA and PEG (MW=400) were dissolved in toluene in the presence of PTSA and the mixture was refluxed while distilling out the formed azeotrope, for 4 days. TLC has confirmed the formation of one major product and a residual amount of the starting material. The major product was identified by ¹H-NMR (data not shown).

Preparation of PPA-JEFAMINE2000-NH₂:

PPA-JEFAMINE2000-NH₂ was prepared as depicted in Scheme 5.

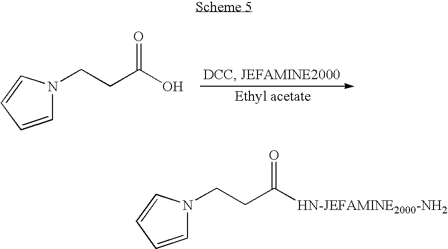

JEFAMINE2000 (O-(2-aminopropyl)-O′-(2-methoxyethyl)-O′-(2′-methoxy ethyl)propylene glycol 2000, 10 grams, 5 mmol) was dissolved in 150 ml of ethyl acetate. While stirred, PPA (0.7 grams, 5 mmol) and DDC (1 gram, 7 mmol) were added thereto. The mixture was stirred at room temperature for 72 hours. Throughout this time a white DCU precipitate formed. The precipitate was filtered off and washed with two 20 ml fractions of ethyl acetate. The ethyl acetate fractions were collected and evaporated to dryness. The obtained yellowish gum was allowed to cool to room temperature and after a while solidified. The product was then purified by gel filtration and identified by ¹H-NMR (data not shown).

Preparation of Bis-Pyrrole-PEG220:

Bis-pyrrole-PEG220 was prepared as depicted in Scheme 6.

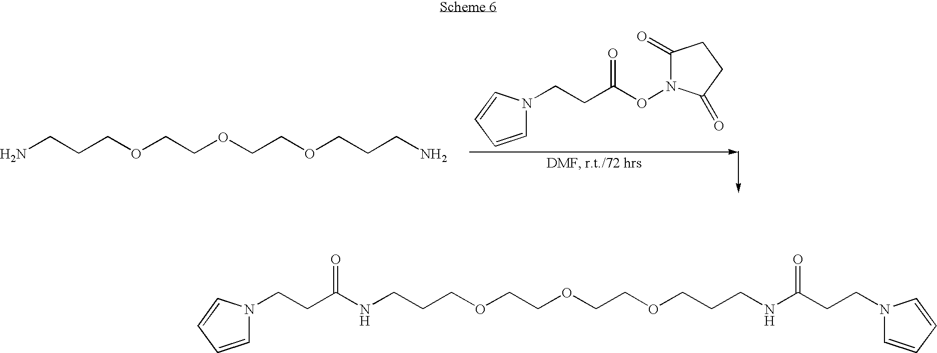

H₂N-PEG₂₂₀—NH₂ (1 gram, 4.54 mmol) was dissolved in 50 ml DMF. Then, PPA-NHS (2.14 grams, 9 mmol) dissolved in 20 ml DMF was added dropwise. The mixture was stirred at room temperature for 48 hours. Upon completion of the reaction the solvents were removed to dryness under reduced pressure. The bis-pyrrolylated residue was separated between 50 ml double distilled water (DDW) and CH₂Cl₂ and was extracted to 3×70 ml CH₂Cl₂. The organic fractions were dried on anhydrous sodium sulfate and the solvent was removed under reduced pressure. The residue was then purified on column chromatography and the final product was identified by ¹H-NMR (data not shown).

Preparation of N-Alkylated Pyrroles—General Procedures:

In a typical reaction, pyrrole was first reacted with NaH, K or butyl lithium to obtain alkali pyrrole derivatives. These were reacted with equimolar amount of acyl halide or haloalkyl as previously described (E. P. Papandopoulos and N. F. Haidar, Tetrahedron Lett. 14, 1721-23, 1968; T. Schalkhammer et al. Sensors and Actuators B, 4, 273-281; S. Cosneir, Electrtoanalysis 1997, 9: 894-902 and references therein). Finally, the pyrrole alkali salt was conjugated with monobromo methoxy Polyethylene glycol (PEG) of various lengths (MW=200, 1000, 4,000 grams/mol, compounds 1, 2 and 3, respectively).

An alternative general procedure sodium hydride was used for in situ preparation of the pyrrolide anion, as depicted in Scheme 7 below.

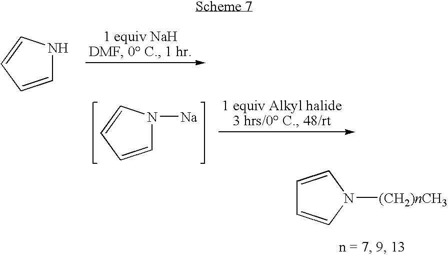

Thus, freshly distilled pyrrole (1 ml, 15 mmol) was dissolved in 30 ml of dry DMF under calcium chloride tube and the solution was cooled to 0° C. in ice cold bath. 1 equivalent of sodium hydride was added as an oil dispersion in fractions to the stirred solution. Immediately, gas evolution was noticed and the mixture was gently stirred for 60 minutes. To the cooling yellowish foam, an alkyl halide (1 equivalent, e.g., octyl iodide, docyl iodide, C₁₄-bromide) dissolved in 20 ml dry DMF, was added dropwise, and the mixture was stirred at 0° C. for additional 4 hours. Thereafter, the mixture was allowed to warm to room temperature, and was left for 48 hours. The DMF was removed to dryness under reduced pressure and the product was extracted from 100 ml DDW to 4×100 ml CH₂Cl₂. The organic fractions were collected and dried over anhydrous sodium sulfate. The organic solvent was then removed to give a brown oil. Purification was performed by distillation under vacuum at 180° C.

Preparation of Derivatives and Analogs of 1,2-di(2-pyrrolyl)ethenes—General Procedure:

1,2-Di(2-pyrrolyl)ethenes and related compound were prepared according to Hinz et al. [Synthesis, 620-623 (1986)], as depicted in Scheme 8.

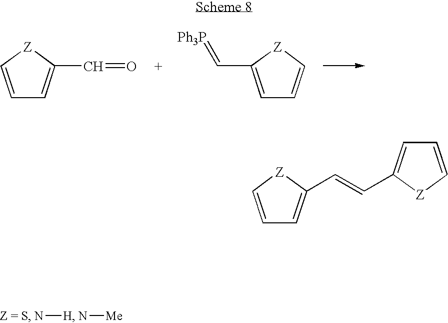

Thus, 1,2-Di(2-pyrrolyl)ethenes and related compounds were prepared via the Wittig reaction between commercially available 2-thiophen carboxyaldehyde or 2-(N-alkylpyrrole)-carboxyaldehyde and the corresponding methyl phosphonium salts (prepared via the Mannich reaction of unsubstituted pyrrole) in toluene (10 hours reflux under argon atmosphere). The overall yields were about 70%.

Preparation of 1,1′-di-(2-thienyl or pyrrolyl)-2-alkyl ethylene—General Procedure:

The titled compounds were prepared as depicted in scheme 9.

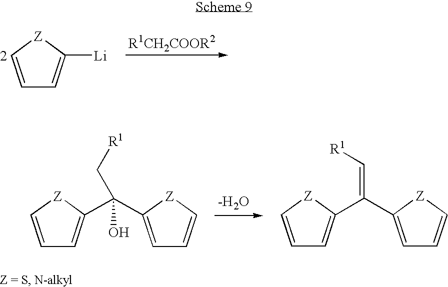

1,1′-Di-(2-thienyl)ethylene was prepared by reacting 2-acetylthiophe with the granger reagent of 2-bromothiophen in dry THF. The product was identified by ¹H-NMR and EI-MS (data not shown).

The Pyrrole analogs were prepared in a similar manner, based on Ramanthan et al. [J. org. chem. 27 1216-9 (1962); and Heathcock et al. [J Heterocyclic chem. 6(1) 141-2 (1969)], via the lithiation of N-Alkylpyrrole in dry hexane or THF with TMEDA at room temperature, followed by disubstitution of the corresponding ester.

The conjugated product was easily obtained in dilute hydrochloric acid.

Further derivatization may be achieved via esterification of the hydroxyl with various carboxylic acids, using known procedures.

Coupling of Thienyl, Furanyl, and N-Alkyl Pyrrole Derivatives—General procedure:

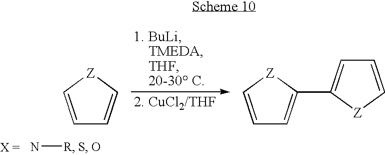

Coupling of the 2-lithium derivative of both thienyl and furanyl derivatives and N-Alkyl pyrrole was performed as depicted in Scheme 10. The various coupling products were easily obtained in relatively good yields (about 70%) using CuCl₂, although other reagent such as NiCl₂ can also be used, as proposed in the literature [chem. Ber 114 3674 (1981)].

Preparation of Electropolymerizable Thienyl and Pyrrolyl Monomers:

1,4-di(2-thienyl)-1,4-butandiol was prepared using Stetter reaction [Stetter, H; Angew chem. 88, 694-704 (1976)] according to Wynberg [Wynberg et al synthetic comm. 1 14(1) (1984)] in a 75-80% yield.

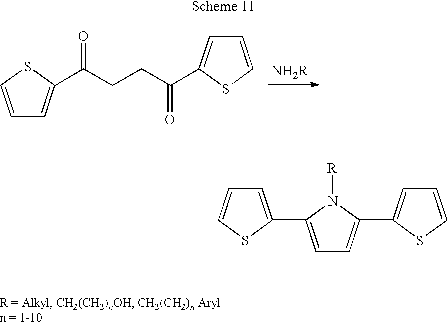

1,4-di(2-thienyl)-1,4-butandiol was then reacted with the corresponding amine to prepare the 2,5-di(2-thienyl)N-alkyl pyrrole via the Paal-Knore reaction [Cava et al Adv materials 5 547 (1993)], as depicted in Scheme 11. The N-alkylhydroxy derivative was conjugated to various carboxylic acid via esterification prior to polymerization.

Preparation of 3-alkyl-(N-Methylpyrrole) Derivatives:

The preparation of 3-alkyl-(N-Methylpyrrole) derivatives is depicted in Scheme 12.

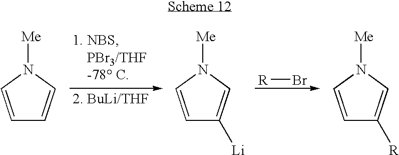

Alkyl pyrrole was selectively brominated with N-bromosuccinimide and PBr₃ in THF according to Dvorikova et al [Dvorikova et al. Synlett 7 1152-4 (2002)]), and was then reacted with BuLi in THF at −78° C. The product was obtained through a reaction with the alkyl halide.

Preparation of the Thienyl and N-Alkyl Pyrrolyl Via Dilithiation:

The preparation of 2,5-di-(2-thieny)-N-alkyl)-pyrrolyl) is depicted in Scheme 13.

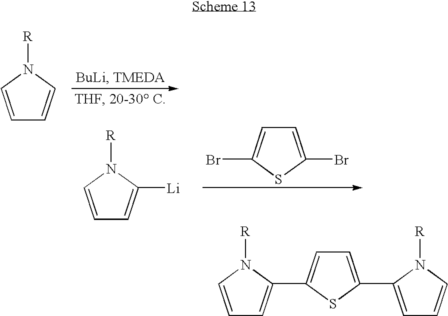

The N-alkyl modified pyrrole was lithiated and the resulting 2-lithium pyrrole derivative was further reacted with 2,5-dibromothiophen.

Preparation of Thienyl and di(N-alkyl)pyrrolyl Dimethanol Oligomers:

The preparation of thienyl and di(N-alkyl)pyrrole dimethanol oligomers is depicted in Scheme 14.

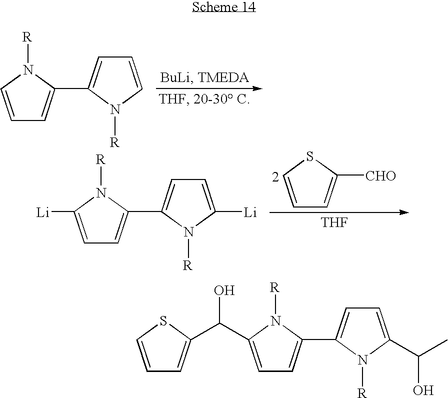

The bis-pyrrole compound (obtained as described in scheme 10 above) was lithiated and the resulting lithiated bis-pyrrole was reacted with an equimolar amount of the corresponding aldehyde. The reaction was carried out according to the procedure described in the literature for reactions of lithium derivatives with aldehyde and ketones in THF under inert conditions [Cava et al Adv materials 5 547 (1993)].

Similar furanyl, pyrrollyl and di(N-alkyl)pyrrole dimethanol oligomers were also prepared using the same process.

Preparation of 2-Alkyl Pyrrole Derivatives—General Procedure

Terminal N-Alkyl pyrrole having alkyl and aryl groups in the alpha position were designed as terminators for the electrochemical polymerization and control of the molecular weight distribution (MWD) of the polymer. These compounds were prepared as depicted in Scheme 15, based on the procedure described in Synthetic Comm. 12(3) 231-48 (1982).

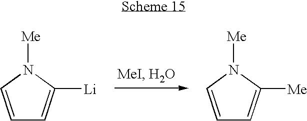

The 2-lithium derivative of N-alkyl pyrroles, such as N-methylpyrrole, was reacted with alkyl or aryl Iodide in Hexane or THF, followed by hydrolysis.

Preparation of N-Alkyl pyrrole-2-carboxylic Acid Derivatives—General Procedure

The preparation of N-Alkyl pyrrole-2-carboxylic acid derivatives is depicted in Scheme 16.

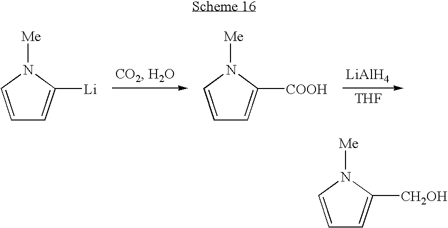

CO₂ powder was added to the 2-lithium derivative of different N-alkyl pyrroles (such as Me, Butyl, hexyl, octyl) at −40° C., to −30° C., followed by addition of water [Jorgenson, org reaction 18 1 (1970)]. The reduction product of the 2-(N-alkyl pyrrole) carboxylic acid was reduced to the corresponding alcohol by LiAlH₄ in THF. The product was identified by ¹H-NMR (data not shown).

The alcohol was attached via esterification to poly acrylic or poly lactic acid to form a pyrrole modified monomer.

The 2-(N-alkyl pyrrole) carboxylic acid was reacted with various PEG molecules to form the corresponding PEG-dipyrrole.

Preparation of N-(3-hydroxy Propyl)pyrrole Derivatives—General Procedure:

N-(2-carboxyethyl)pyrrole, prepared as described above, was reduced by LiAlH₄ in dry THF in a 80% yield, using known procedures. The product was purified by distillation and identified by ¹H-NMR, and EI-MS (data not shown).

The hydroxy pyrrole derivative was attached via esterification to poly acrylic and poly lactic acid to form a pyrrole modified monomer.

Preparation of Pyrrole Conjugates of Modified Carboxylic Acids Containing Amino Groups—General Procedure:

In order to allow the conjugation of carboxylic acids modified by amino containing active agents, to the amino pyrrole, a spacer of glutaraldehyde was used.

In a typical reaction, N-(3-aminopropyl)-pyrrole is first reacted with excess glutaraldehyde to form an imine, which is then reacted with the modified carboxylic acid containing the amino group/s to form a second imine bond. Reducing the imine bonds by NaBH₄ results in stable amine bonds. The advantage of using the imine-aldehyde-amine reaction is that it is carried-out in an aqueous solution in high yields.

Preparation of Pyrrole Conjugates of Modified Carboxylic Acids Containing Saccharide or Polysaccharide—General Procedure:

To allow the conjugation of carboxylic acids modified by saccharide-containing, or polysaccharide-containing agents, to the amino pyrrole, the saccharide is first oxidized to form aldehyde bonds which are then reacted with the aminopropyl pyrrole to form polymerizable pyrrole saccharide derivatives.

Preparation of Pyrrole Conjugates of Modified Carboxylic Acids Containing Hydroxy Groups—General Procedure:

To allow the conjugation of carboxylic acids modified by hydroxy containing active agents, to the amino pyrrole, the amino pyrrole is first esterified using the common activating agents, such as carbodiimides.

Alternatively, the hydroxyl group on the active agents is first conjugated to an amino acid or a short peptide via an ester bond, resulting in an amino or imine derivative thereof, which is then conjugated to the pyrrole either through an amidation reaction, using carbodiimide as a coupling agent, or through an imine bond when using an aldehyde containing pyrrole.

In a typical reaction, amino terminated PEG2000 was reacted with 1.3 equivalents of carboxyethylpyrrole in DMF using DCC as a coupling agent at room temperature for 3 days. The product was isolated by evaporating the DMF to dryness and triturating the residue in diethyl ether. The conjugation yield was over 90% as determined by mass-spectrometry and ¹H-NMR analysis

Preparation of Pyrrole Conjugates of Long Aliphatic Carboxylic Acids —General Procedure:

ω-carboxyalkylyrrole derivatives with longer aliphatic chains are synthesized according to Schuhmann (in Diagnostic Biosensor Polymers, A M Usmani and N. Akmal, eds. ACS Symposium Series 1994, 226, 110, Electroanalysis, 1998, 10, 546-552).

### Example 2

### Preparation of Nanoparticles

Various methods have been described in the literature for the formulation of nano- and microparticles having hydrophilic surface such as PEG chain or polysaccharide chains on the surface (see, for example, R. Gref, et al., Poly(ethylene glycol) coated nanospheres, Advanced Drug Delivery Reviews, 16: 215-233, 1995).

In a preferred method, hydrophilic-hydrophobic molecules having functional groups as part of the hydrophilic side are prepared, such that when the molecule is used for the preparation of particles in a mixture of organic-aqueous solvents, the hydrophilic side chain will remain on the surface towards the aqueous medium. For example, PLA-PEG block copolymer having amino groups on the PEG end chain, can be formulated into particles by a solvent evaporation method using PLA and optionally drug solution in an organic solvent dispersed in aqueous solution, to thereby form particles with PEG chains onto the particle surface that have amino functional groups available for further reactions or interactions.

In a representative example, PLA-PEG-amine copolymer (PLA chain MW of about 3,000 D and PEG chain MW of about 1,000 D) was added to a dichloromethane solution of PLAs of various molecular weights, ranging from 3,000 to 50,000 D (10% w/v), at a ratio of 1:10 per PLA in the solution. The resulting clear solution was added drop-wise to a 0.1M phosphate buffer solution pH 7.4 with high-speed homogenization to form a milky dispersion. The mixing was continued for a few hours at room temperature until all solvent was evaporated. The resulted dispersion contained spherical particles of a particle size in a micron range with PEG chains on the surface, as was determined by the ¹H-NMR spectrum of particles dispersed in deuterated water (data not shown). The presence of surface amino groups was determined by reaction of the particles with FITC, a reagent that renders the particles fluorescent. Using the above procedure, drugs such as paclitaxel can be incorporated in the particles by adding the drug to the PLA solution prior to its addition to the aqueous medium for particle preparation. The amount of drug incorporated in the particles can be from about 1% w/w to about 50% of the polymer weight.

Nanoparticles having pyrrole derivatives bound to the surface and available for electropolymerization were prepared as follows: bromo-PEG2000-hydroxyl was reacted with pyrrole to obtain N-Pyrrole-PEG2000-OH, which was then polymerized with lactide using stanous octoate as catalyst. The block copolymer was then mixed with poly(lactide) and PEG-PLA in a chloroform solution. This solution was added dropwise to a stirred buffer solution (0.01M phosphate pH7.4) to form nanoparticles with PEG-pyrrole on the surface available for electropolymerization.

### Example 3

### Electropolymerization

Pre-treatment of stainless steel (SS) surfaces: SS surfaces were pre-treated prior to electropolymerization thereon, in order to improve their surface properties and provide a better adherence of the polymer thereto.

The adhesion factor on SS plates was measured with cross-cut adhesive tape following D-3359-02 ASTM standard test for SS.

New pre-treatment procedures were developed, and are presented in Table 1 below.

TABLE 1

| Substrate | Pretreatment |
|---|---|
| SS 316 | Manual polish using 4,000 grit sand paper, until |
| plates | shines, rinse in acetonitrile. |
| SS 316LVM | Dip in 40% HNO₃ for 10 minutes at room temperature, |
| plates | rinse with DDW, sonication in DDW and then in acetone |
| | for 10 minutes each. |
| SS 316LVM | Dip in 40% HNO₃ for 10 minutes at room temperature, |
| stents | rinse with DDW, sonication in DDW and then in acetone |
| | for 10 minutes each. |
| | Sonicate or shake for 15-40 minutes in acetonitrile |
| | or ethanol with Carborundum, mesh size 220-1000, or |
| | mixtures of. The temperature was 25-65° C. Rinse |
| | in DDW and in acetone. |

In a typical experiment a SS 316 plate was manually polished using 4,000 grit sand paper, until the plate looked shiny as a mirror. Then it was rinsed with acetonitrile and subjected to electropolymerization with a pyrrole derivative. The best adhesion between the polymer and SS surface was obtained with lower Cr/Fe ratios and with smoother surface topographies. The bulk Cr/Fe ratio is around 0.3, the surface of the metal alloy may contain as high as 1.65 ratio, which protects it against corrosion. When the SS 316 plates were manually polished as described in Table 1 above, the Cr/Fe ratio decreased from 1.09 to 0.38. Consequently the average adhesion factor of eight different coatings increased from 0.2 to 0.8.

In stents, Carborundum treatment procedures, as described in Table 1 above, gave best adhesion of the polymer. The Cr/Fe ratio following this treatment decreased from 0.67 to 0.38. In a typical experiment conducted with a stent, the stent was sonicated with a 1:1:1 mixture of 220, 500 and 1,000 carborundum powder in ethanol for 40 minutes and the temperature rose to about 65° C. Then the stent was rinsed with a pressurize DDW to remove all powder from its surface. The stent was finally rinsed in acetone to dry it from DDW.

Different mixtures containing carborundum of different mesh size were used to clean the stents prior to polymerization. The mixtures were agitated using a vortex and then washed with DDW, and acetone. Coating of the stents was carried out in BuOPy:PPA 10:1 in acetonitrile 0.1M TBATFB. The results are presented in Table 2 below.

TABLE 2

| | | | Visual | Visual | Delamination |
|---|---|---|---|---|---|
| No. of | | Time | inspection | inspection | upon manual |
| Stents | Mixture | (minutes) | after treatment | after coating | rubbing |
| 4 | Carborundum | 20 | Slightly | Slightly | 50% |
| | 1,000 in | | poked | poked | |
| | Hexane | | | | |
| 1 | Carborondum | 20 | Slightly | Slightly | total |
| | 1,000 in | | poked | poked | |
| | H2SO4 with | | | | |
| | K₂Cr₂O₃ | | | | |
| 1 | Autosol | 20 (50° C.) | Shiny | STD | total |
| Thick | Autosol | polished | Shiny | STD | Minimal |
| polished | | | | | delamination |
| plate | | | | | |
| 2 | Carborondum | 20 in | Slightly | STD | Very slight |
| | 220 in AN | sonicator | scratched | | delamination |

These results indicate that the Carborondum mesh 220 is superior to all the other tested pre-treatments in promoting the adhesion of the polymer to the stent. The sonication procedure used in this technique enables to carry out the pre treatment procedure in large numbers (10 in one container).

Electropolymerization of Stent with Various N-Substituted Pyrroles:

Using the Carborondum mesh 220 pre-treatment described above, the performance of electropolymerized polymers formed by electropolymerization in the presence of various N-substituted pyrroles, in addition to the BuOPy:PPA 10:1 mixture was tested. The stents were sonicated, prior to electropolymerization, in acetonitrile (AN) with carborundum 220 mesh for 15 minutes and then washed with DDW and acetone, and dried over a stream of nitrogen. The stents were manually rubbed to inspect the adhesion of their coating and expanded to 3 mm OD with a balloon in DDW.

Electropolymerization of stents was carried out in mixtures of N-alkyl and 2-acetyl pyrroles, in acetonitrile with 0.1M TBATFB, as detailed below. The mixtures consisted of 0.07 M BuOPy (butyl ester pyrrole), 0.01 M PPA and 0.02 M pyrrole or N-alkyl pyrrole. The results are presented in Table 3 below.

TABLE 3

| | No. of | Delami- | | |
|---|---|---|---|---|
| Monomer | stents | nation | After expansion | Comments |
| None* | 2 | 1 delami- | >10 μM tears on | Only 1 |
| | | nated | almost every | expanded |
| | | | junction (50%) | |
| 2-acetyl-pyrrole | 3 | partial | 5 μM tears | 2 expanded, |
| or unmodified | | | not on every | one |
| pyrrole | | | junction (50%) | compressed |
| | | | | (to 1.1 mm). |
| N-methyl- | 3 | none | Essentially no | Stable, |
| pyrrole | | | tears | adherent, |
| | | | | uniform |
| N-hexyl-pyrrole | 2 | none | Essentially no | Stable, |
| | | | tears | adherent, |
| | | | | uniform |

*0.1M BuOPy and 0.01M PPA for reference.

It should be noted that in these experiments, stents were expanded to 2.7-2.95 mm. The stents were all expanded symmetrically and therefore it is suggested that the polymer formed in the presence of the 2-acetyl and both N-alkyl pyrroles is more flexible than that prepared from the BuOPy:PPA, 10:1 mixture.

### Electropolymerization:

Electropolymerization on SS Plates:

Electrochemical measurements were conducted with an 630B electrochemical analyzer (CH Instruments), using a single compartment three electrode glass cell. The reference electrode was an Ag|AgBr wire that was used in organic media. The latter has a potential of 0.448 V vs. ferrocene-ferrocenium (Fc/Fc⁺). A 6 mm diameter graphite rod was used as an auxiliary electrode. A typical polymerization cell setup is presented in FIG. 1.

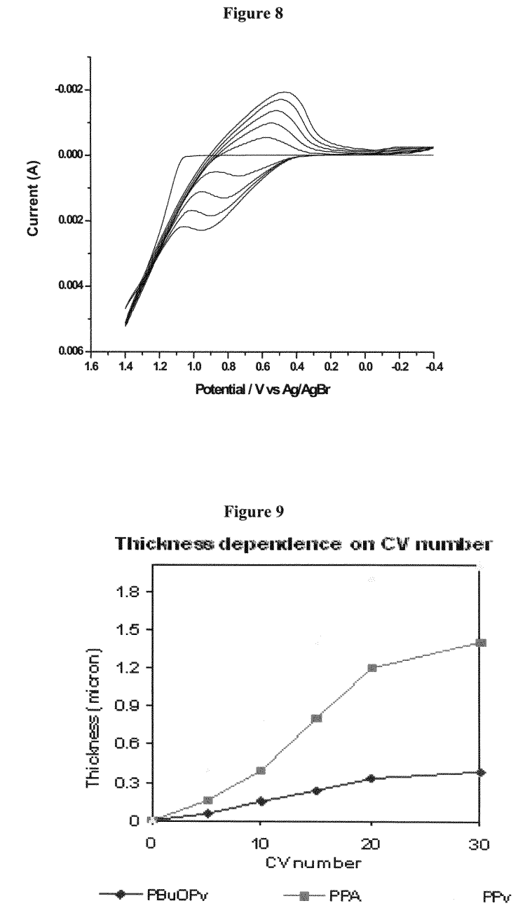

In a typical experiment pyrrole was electropolymerized on a stainless steel plate (40×9 mm²) in an acetonitrile solution containing 0.1 M distilled pyrrole derivative monomer/s and 0.1 M tetrabutylammonium tetrafluoroborate (TBATFB) using cyclic voltammetry. A potential sweep between −0.8 to 1.2 V vs. Ag|AgBr was typically applied (10 cycles unless otherwise mentioned). FIG. 8 presents a typical cyclic voltametry diagram. Graphite rod was used as an auxiliary electrode while Ag|AgBr was used as the reference electrode. The latter, which has a potential of 0.448 V vs. ferrocene-ferrocenium (Fc/Fc⁺) (14), was found to have a much more stable potential in the organic media than the commonly used Ag|AgCl wire.

In addition to unmodified pyrrole solution, other monomeric solutions were prepared and used the electropolymerization solution: 100% pyrrole propanoic acid, 100% pyrrole butyl ester, 100% PEG400 dipyrrole, and a mixture of 50:50 pyrrole propanoic acid:pyrrole butyl ester. The electrochemical conditions were: initial potential −0.4 V, highest potential 1.6 V, final potential −0.4 V. The solution had monomer concentration of 0.1 M, with 0.1 M of TBATFB in 10 ml of acetonitrile. For each solution 5,10,15,20 and 30 CV were sampled.

The changes in parameters like the range of potential sweep, monomer concentration and number of cycles varies with the pyrrole different derivatives.

FIG. 9 presents the thickness obtained with each of the tested solutions as a function of the CV number. The results show that poly(pyrrole propanoic acid) and poly(pyrrole butyl ester) keep linearity up to 20 CV while at 30 CV the linearity is failed. The mixed solution of pyrrole propanoic acid and pyrrole butyl ester has a film thickness value that is between that of the PPA and the PBuOPy, such that at 20 CV the thickness is 0.7 μm.

The results presented in FIG. 9 clearly demonstrate that the polymerization rate and final polymer thickness are reduced dramatically as the length of the chain attached to the N-position of the pyrrole is higher.

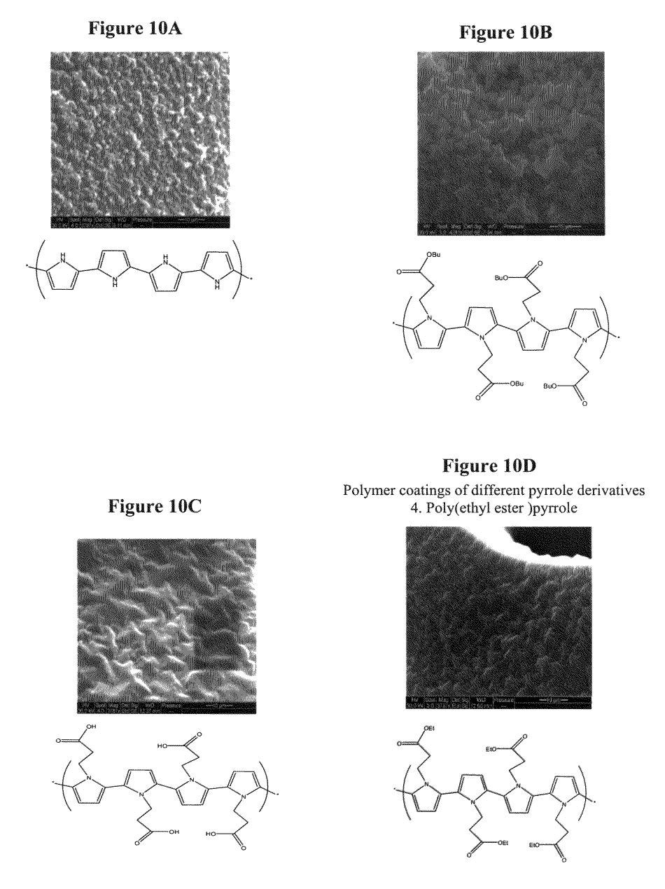

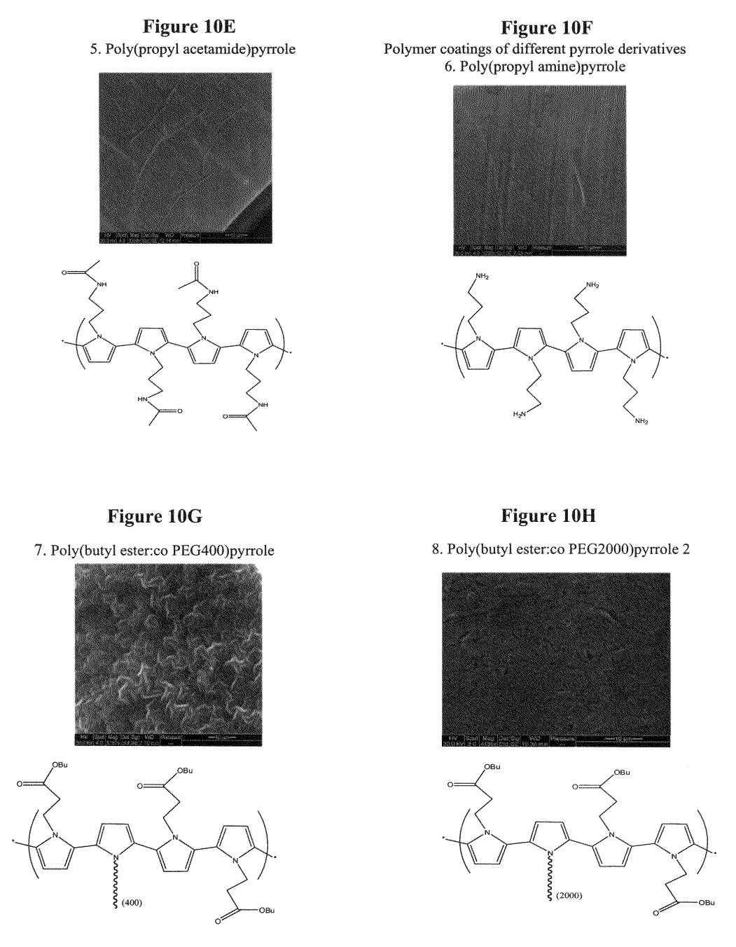

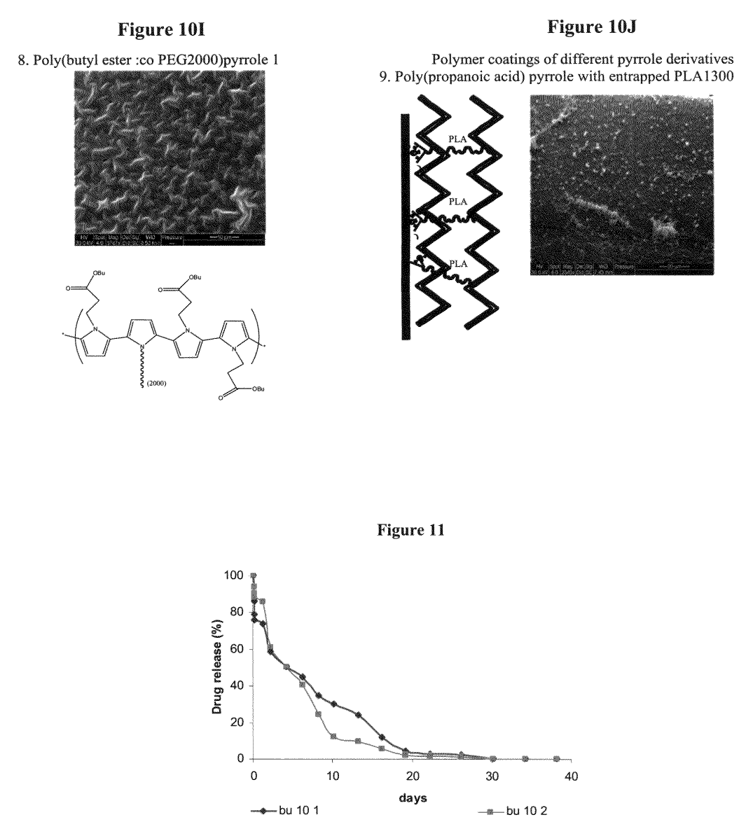

FIG. 10 presents SEM measurement of stainless steel surfaces electropolymerized in the presence of various monomers and clearly show uniform full coverage of the metal surface.

Electropolymerization in Stents:

General Procedure I: Coating of stents by electropolymerization was carried out in a three electrode cell, in which the stent, connected to the electrical circuit through a 316L stainless steel screw, acted as the working electrode. The auxiliary electrode consisted of a platinum wire or a glassy carbon rod, and the reference electrode was a silver wire coated with silver bromide (0.448 Volts vs Ferrocene).

The working electrodes were polished first with 240, 600 and 2000 grit emery paper (Buehler), followed by fine polishing by alumina paste (1 and 0.05 um) on a microcloth polishing pad. The electrodes were then washed and sonicated for 15 minutes in acetonitrile, and were dried at room temperature prior to the electrochemical polymerization.

The polymer is deposited on the stent by applying either cathodic or anodic voltages. The coating consisted of a polymer formed by electrodeposition in one of the following methods:

(i) Amperometry, in which the potential is kept constant for a determined amount of time. A typical experiment consists of applying 20 microAmpers for 3 minutes;

(ii) Galvanostatic, in which the current is kept constant for a determined amount of time. A typical experiment consists of applying 1.6 V vs Ag/AgBr for 3 minutes; and

(iii) Cyclic or pulse voltammetry, which allow the potential to be cycled between two values, or to be applied in pulses. A typical experiment consists of applying 5-20 cycles at a rate of 100 mV/sec from −0.4V to 1.6 V vs Ag|AgBr. An example of the pulse method is alternating anodic and cathodic pulses for different periods of time. In this way a mixture of two monomers, one that undergoes oxidative polymerization and the other undergoes cathodic polymerization, may be deposited on the same electrode surface.

The exact current or potential values are chosen according to the properties of each monomer used.

Direct current (dc) cyclic voltammetry and chronoamperometric experiments are performed with an EG&G Princeton Applied Research potentiostate/galvanostat interfaced to a PC.

General Procedure II: A single glass compartment kept at 25° C. was used. The reference electrode was a saturated calomel electrode (SCE) and a counter electrode platinum wire. Working electrodes were connected to a typical stent material. The electrolyte solution used in these experiments was a 0.1M sodium phosphate buffer solution containing 0.1M NaCl at pH=7.0, or containing 0.1 M Bu₄NBF₄ in CH₃CN solution. The pyrrole polymer was deposited at the stent wire by electrochemically oxidizing an electrolyte solution containing 0.1M freshly distilled pyrrole and known amounts of pyrrole derivatives. The oxidation potential was performed at 0.7V versus SCE until the amount of charge passed was 10 mC. The resulting coated polymer electrode was rinsed thoroughly in distilled water. Typical pyrrole compositions included a mixture of heparin-pyrrole derivative: PEG-pyrrole derivative: pyrrole, at a molar ratio of 1:1:8.

Exemplary procedures: Using general Procedure I described above, electropolymerization was performed in acetonitrile or DMF with 0.1M TBATFB using the following substrates:
A single monomer listed in Table 4 below;
A mixture of 2 or more of the monomers listed in Table 4, at various ratios;
A mixture of one or more monomers, and one or more of the surfactants or additives listed in Table 5 below;
A mixture of monomers for a double-layered coating, as detailed hereinbelow. A first layer is formed by cycling the potential of the stent in one monomer solution, removing the stent from the solution and immersing it in a new solution of a different monomer to form the next layer;
A mixture of monomers for two-step polymerization: a pyrrole derivative is electropolymerized and chemical polymerization is then performed for polymerizing thereon a second polymer, as detailed hereinbelow;
A single monomer for two-step polymerization: a pyrrole derivative is electropolymerized and chemical polymerization is then performed for polymerizing a functional substituent of the pyrrole;

An example of the latter is Pyrrole-Et-COO—CH₂—CH═CH₂ presented hereinbelow. This monomer is anodically electropolymerized through its pyrrole group, leaving a surface covered by allylic end group. These allylic groups may be further polymerized using an initiator such as AIBN, resulting in a highly crosslinked polymer, forming a “sleeve” on the stent surface.

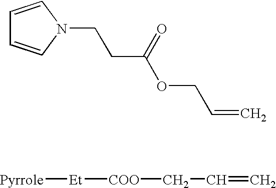

Radical polymerization using initiators was carried out by overnight dipping the electrocoated plate in a solution containing the initiator at 0.5-1% w/w in acetonitrile or THF, at 50-60° C.

TABLE 4

| | |
|---|---|
| Pyrroles | Pyrrole |
| | PPA |
| | 2-acetyl-Pyrrole |
| | Pyr-Et-COO—R |
| | (R = methyl, ethyl, isopropyl, butyl, isobutyl, |
| | secbutyl, amyl, cyclohexyl, octyl, 2-metyl 2-propenyl, |
| | metoxybenzophenonyl) |
| | N-R-pyrrole (R = methyl, isopropyl, propyl, hexyl. |
| | octyl, dodecyl) |
| | 1-bromobutane-4(1-pyrrole) |
| | 1,2,6-tri(N-propanoyl pyrrole)-hexane |
| | 1,1′,1″,1″′-tetra(N-propanoyl pyrrole)-methane |
| | MethoxyPEG550 pyrrole |
| | PEG400 dipyrrole |
| Methacrylates | Methylmethacrylate |
| | Laurylmethacrylate |
| Bi-functional | Pyrrole-Et-COO—CH₂—CH═CH₂ |
| Thienyl | 1,1′-di(2-thienyl)ethylene |
| derivatives | 3-dimethylamino-1-(2-thienyl)-propanone |
| | 1,4-di(2-thienyl)-1,4-butandiol |

TABLE 5

| Additive/anion | Concentrations |
|---|---|
| PEG (1,000, 2,000, 4,000) | 5%-10% |
| LA(monomers, polymers of | 5%-10% |
| MW = 1000) | |
| RA (monomers) | 5%-10% |
| PVP30, 90 | 10% |
| Triethyl citrate | 10% |
| p-toluenesulfonate | 10⁻⁵-0.1M |
| dodecylsulfonate | 0.01M |
| TBA perchlorate | 0.1M |
| Water | 0.1-20% |

### Example 4

### Electropolymerized Polymers Having Active Agents Covalently Attached Thereto

Bioactive agents (e.g., peptides or proteins) were conjugated to the pyrrole monomers either via amino pyrrole (see, Example 1 above) or via carboxyethyl pyrrole (see, Example 1 above). The conjugate was isolated by gel filtration chromatography or by dialysis.

In a typical reaction, a bioactive agent (for example, heparin) was conjugated to carboxyethyl pyrrole by amide coupling using DEC in Na-HEPES buffer of pH=7.4. The obtained conjugate was separated from the reaction mixture by gel filtration chromatography.

Using the general procedure II described above for electropolymerization on stents, an electrocoating having heparin covalently attached thereto was prepared.

In yet another typical reaction, PPA was reacted with carboxylic acid-containing drugs, other bioactive agent or hydrophilic or hydrophobic residues (e.g., fatty acids), by either of the following procedures:

(i) a direct reaction in DMF using dicyclohexyl carbodiimide (DCC) as a coupling agent; or

(ii) a reaction with a reactive derivative of the carboxylic acid, i.e. acid chloride, anhydride, N-succinimide, or the carboxylic acid.

When amino-PEG-pyrrole was reacted with an aldehyde-containing active agent, an imine (Schiff base bond) was obtained. This biodegradable imine conjugate product was used when designing the controlled-release of the active agent from the pyrrole coating (the release rate being a function of the imine bond degradation). However, when a stable, non-degradable, bond was desired, the pyrrole-imine-drug conjugate was further reduced to the corresponding amine bond using NaBH₄ as reducing agent.

Alternatively, controlled releasable active agents may be incorporated to the electropolymerized film during its formation by adding to the polymerization solution the pyrrole-substituted nanoparticles prepared as described above (see, Example 2), further encapsulating the active agent. The active agent is slow-released from the resulting polymeric coating by via diffusion through the particle matrix and then through the electropolymerized coating. The conjugation methods for binding an active agent like heparin, a steroid or a peptide or protein via a cleavable or non-cleavable bond are adopted from procedures described in: Bioconjugate Techniques, G. T. Hermanson, editor, Academic Press, San Diego, 1996).

Further alternatively, coatings by amino or carboxylic acid pyrrole derivatives were prepared on the stent, and the active agent was conjugated to the already prepared pyrrole film. Deposition of poly(co-carboxyalkylpyrrole) was performed in a potentiostatic pulse regime from a 10 mM monomeric solution in an acetonitrile solution containing 100 mM (Bu)₄NPF₆ as electrolyte salt. A pulse profile consisting of pulses of 950 mV for 1 second followed by a resting phase for 5 seconds was applied to form a thin functionalized polypyrrole layer. In general, 5 pulses were sufficient to cover the electrode surface with a thin polymeric film for covalent binding of an amine containing agent. The coated stent was immersed for at least 10 hours into a 3 mM heparin solution containing 30 mM N(3-dimethylaminopropyl)-N-ethyl-carbodiimide hydrochloride to activate the carboxylic acid groups of the polymer. After rinsing the electrode with ethanol, the second layer was formed on top of the heparin bound layer, by electrochemical deposition of polypyrrole and PEG derivatized pyrrole. This double layer provides a passive protection on the stent by the hydrophilic PEG chains and active protection prolonging the release of the attached heparin for a period of weeks.

### Example 5

### Preparation of Electrocoated Metal Surfaces Loaded With Active Agents

Drug Loading:

Stents were electrocoated with electropolymerized polybutyl ester pyrrole and poly(butyl ester co-propanoic acid)pyrrole (10:1 BuOPy:PPA). The electropolymerization was carried out by applying 5 or 10 CV (cyclic voltammograms). Coating thickness of the samples obtained by 5 CV was 0.4 μm, and by 10 CV was 0.6 μm.

Drug loading on the coated stents was carried out by swelling: the polypyrrole-coated stents were immersed into 20 mg/ml solution of Paclitaxel in acetonitrile for 0.5 hour, and were then air dried. Optionally, after air drying, the stents were immersed in a 20 mg/ml solution of acetonitrile containing 0.01 M of polylactic acid (PLA, 1300) for 5 minutes. Alternatively, the swelling procedure was carried out in other solutions such as ethanol or chloroform solutions, using various concentration of Paclitaxel (e.g., 30 and 40 mg/ml).

Loading of drug was measured by stripping drug off the stent or plate to a 2 milliliter of acetonitrile solution using an ultrasonic bath;

diluting 100 μl of this solution in one milliliter of buffer phosphate solution 0.1 M, pH 7.4 (0.3% SDS); and

analyzing final solution by HPLC to determine the loaded drug concentration.

Table 6 below presents the results obtained while loading the drug on various electrocoated stents.

TABLE 6

| | Total drug |
|---|---|
| Pyrrole derivative | loading(μg/mm²) |
| Poly(butyl ester)pyrrole 20 cv 1 | 0.89 |
| Poly(butyl ester)pyrrole 20 cv 2 | 1.32 |
| Poly(butyl ester)pyrrole 10 cv 1 | 0.85 |
| Poly(butyl ester)pyrrole 10 cv 2 | 1.71 |
| Poly(butyl ester:propanoic acid) pyrrole 20 cv 1 | 0.9 |
| Poly(butyl ester:propanoic acid) pyrrole 20 cv 2 | 1.15 |
| Poly(butyl ester:propanoic acid) pyrrole 10 cv 1 | 1.73 |
| Poly(butyl ester:propanoic acid) pyrrole 10 cv 2 | 1.91 |

Drug Release:

In vitro measurements of the release of the loaded agent were performed by measuring the passive diffusion of the agent into an aqueous solvent such as a phosphate buffer, as follows:

Drug-loaded stents were placed in 1 milliliter of a buffer phosphate solution 0.1 M, pH 7.4 (0.3% SDS) at 37° C., and shaking was performed at set time points. Absorbed Paclitaxel was removed during the first half an hour. At each time point, the drug release concentration was measured by HPLC.

Resulting retention time for Paclitaxel was 7.9-8.4 minutes at 1 ml/minute flow of DDW:ACN (45:55) as a mobile phase.

In an exemplary experiment, stents coated with poly(butyl ester)pyrrole were immersed into 20 mg/ml solution of Paclitaxel in acetonitrile for 0.5 hour, and were then air dried. Other stents were similarly treated and after air drying, were immersed in a 20 mg/ml solution of acetonitrile containing 0.01 M of polylactic acid (PLA, 1300) for 5 minutes.

The drug release was measured as described above and the results are presented in FIG. 11. As can be seen in FIG. 11, with both type of stents, the drug was gradually released over a period of more than 30 days, whereby with stents that were further treated with PLA, the release was slightly slower.

### Example 6

### Multi-Layered Coatings

In this example, the preparation of multi-layered coatings, prepared by using bifunctional monomers and/or by impregnating polymers into or onto electropolymerized polymers, designed to enable loading drugs therein, is described.

To that end, three general approaches were designed and practiced, as follows:

(i) bifunctional monomers, having an electropolymerizable moiety and a chemically polymerizable group, were prepared and subjected to a two-step polymerization process: electrochemical polymerization, followed by a chemical polymerization (e.g., free radical polymerization in the presence of a catalyst);

(ii) electropolymerizable bifunctional monomers having a photoreactive group (PAG) were prepared and subjected to a two-step polymerization process: electrochemical polymerization, which resulted in activated polymer, followed by a chemical polymerization, which is catalyzed by irradiation and induced by the activated polymer, and is performed in the presence of another monomer and/or a drug; and

(iii) electropolymerizable bifunctional monomers having a reactive group were prepared and subjected to a two-step polymerization process: electrochemical polymerization, which resulted in activated polymer, followed by a chemical polymerization, in the presence of a catalyst, and another monomer and/or a drug, in which the reactive group participates.

In addition to the above, multi-layered coatings were also obtained by a simple multi-step polymerization process, which included one or more consecutive electrochemical polymerization processes, optionally followed by impregnation of an additional non-electropolymerized polymer, as described hereinabove.

In each of the above procedures, the final multi-layered stent can be immersed in a drug solution for drug loading. Alternatively, the drug can be loaded during one or more of the chemical polymerization processes by adding the drug to the polymerization solution.

Two-Step Polymerization Route Via Chemically Active Groups of Pyrrole Derivatives:

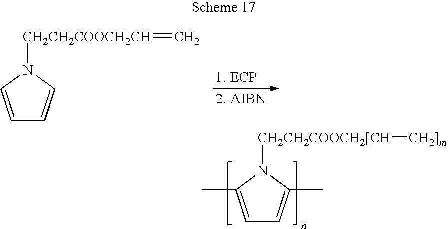

Vinyl derivatives of pyrrole were prepared by reacting N-(2-carboxyethyl)pyrrole with allyl alcohol to yield the corresponding allyl ester in 60% yield, or by reacting N-(2-carboxyethyl)pyrrole with acryloyl chloride in dichloromethane and in the presence of triethylamine [as described in Min Shi et al., molecules 7 (2002)]. The vinyl pyrrole derivative was electrochemically polymerized via the 2 and 5-positions of the pyrrole unit, resulting in a polymer having free vinyl groups attached thereto. This polymer was further polymerized in the presence of AIBN or benzoyl peroxide as initiators for free radical polymerization of the monomer.

This general approach was described, for example, for the free radical polymerization of N-vinyl pyrrole with AIBN, follows by second polymerization with FeCl₃ [see, for example, Ruggeri et al Pure and appl chem. 69 (1) 143-149 (1997)].

Preparation of Electropolymerized Polymers Having Photoreactive Groups Attached Thereto:

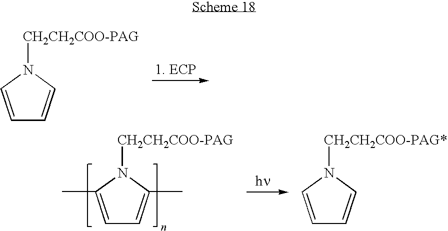

An electropolymerizable pyrrole monomer having a benzophenone derivative, as an exemplary photoreactive group, was prepared by an esterification reaction between N-(2-carboxyethyl)pyrrole and a benzophenone reactive derivative, such as 2-hydroxy-4-methoxy-benzophenone, in toluene, using para-toluene sulfonic acid as a catalyst, and Na₂SO₄ and MgSO₄ as desiccants. Following electrochemical polymerization, polypyrrole having benzophenone groups attached thereto was obtained. This polymer was activated by irradiation, to allow an additional, chemical polymerization process, which is induced by the activated groups.

Polyacrylate-Containing Multi-Layered Coatings:

Double-layered drug-loaded polyacrylate-containing coatings on stents were prepared is order to improve the mechanical properties of the polypyrrole coating and/or to improve the total loading and to optimize the releasing profile from the stents coated by polypyrrole derivatives.

Such double-layered coated stents were prepared using two methods as follows:

Method 1: polypyrrole-coated stents were obtained as described above, using a mixture of 1:7:2 (molequivalents) PPA, PPA butyl ester and PPA hexyl ester as the electropolymerization solution and were thereafter immersed in solution of 40 mg/ml paclitaxel and 1% polymethyllauryl (2:3) methacrylate in chloroform for one minute. Then, the stents were dried and immersed again for one minute in the same solution, and were finally dried again. Thereafter, the dry stents were immersed in a solution of 1% polymethyllauryl (2:3) methacrylate in cyclohexane for 20 seconds.

Total drug loading was 85-100 μg on each stent.

The coating thickness was about 0.8 μm.

Method 2: polypyrrole-coated stents were obtained as described above, using a mixture of 1:7:2 (molequivalents) PPA, PPA butyl ester and PPA hexyl ester as the electropolymerization solution and were thereafter immersed in a solution containing 30 mg/ml paclitaxel in ethanol for 30 minutes. Stents were thereafter immersed in a solution containing 40 mg/ml paclitaxel and 1% polymethyllauryl (2:3) methacrylate in chloroform for one minute, and dried. The dry stents were then immersed in a solution containing 1% polymethyllauryl (2:3) methacrylate in cyclohexane for 20 seconds.

Total drug loading was 85-110 μg on each stent.

The coating thickness was about 0.8 μm.

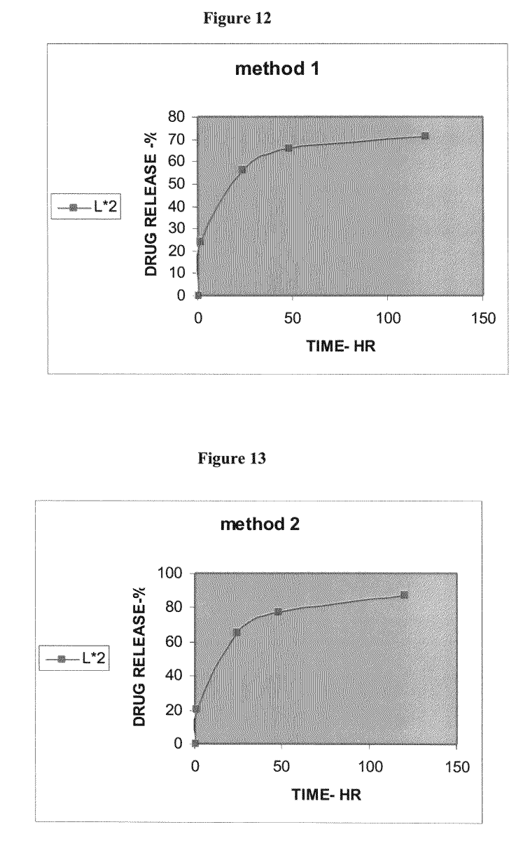

FIGS. 12 and 13 present the drug release profile from stents prepared by method 1 (FIG. 12) and method 2 (FIG. 13). As can be seen in FIGS. 12 and 13, using both stents, the drug was slowly released over a period of more than 100 days. Slower drug release was observed in stents prepared by method 2.

Poly(Allyl Ester) Pyrrole Coating Modification with Lauryl Methacrylate and PETMA, on Stents:

Bifunctional monomers such as the allyl ester derivative of pyrrole described hereinabove, which contains pyrrole units were used to obtained stents coated with poly(allyl ester)pyrrole. The coating thickness was 0.4 μm. Modification of the stent surface by another polymerization of an acrylate monomer was then performed as follows:

Polymerization of Lauryl Methacrylate (Benzoyl peroxide (BP) as initiator): To a lauryl methacrylate (LM) monomer solution (either neat or 50% LM in DCM), 1% w/v of BP per monomer was added. The allyl ester polypyrrole-coated stent was immersed in the solution for 5 seconds. Then the stent was dried to remove excess of the LM solution and inserted to an empty small glass vial under stream of nitrogen for some minutes. The vial was closed and heated to 70° C. for 5 hours. After the reaction was completed the stent was rinsed with methanol and expanded. A uniform coating was obtained.

Crosslinked polymerization of Lauryl Methacrylate with PETMA (pentaeritritoltetrametacrylate) (BP as initiator): Using the same procedure as above, a cross-linked polyacrylate coating was obtained by adding to the acrylate monomer solution 1% w/w PETMA as a cross-linking agent.

Polymerization of PETMA (BP as initiator): Using the same procedure as above, a cross-linked polymer coating was obtained by using a solution of 50% PETMA in DCM as the monomer solution.

Polymerization in aqueous medium: each of the procedures described above was performed by immersing the stent in the monomer solution, drying the stent and immersing the resulting stent in water under nitrogen stream. Then 0.25% of Na₂S₂O₅, 0.25% of FeH₈N₂O₈S₂ and Na₂S₂O₈ were added and the mixture was stirred for 5 hours. The stent was then rinsed with water and expanded.

Each of the electropolymerization processes described hereinabove (e.g., in Examples 3-5), can be performed on stents or other implantable devices, as well as on certain parts of the device. For example, the inner part of a metal stent can be protected from electropolymerization coating by inserting the stent onto an inflated balloon or a soft or rigid rod, thus limiting the access of the electropolymerization solution to the inner side of the stent. Likewise, the inner part can be electrochemically coated without coating the surface, by covering the outer part with a balloon or a soft cover. A device can be coated by various coating layers to allow the desired properties. For example, the initial polymerization layer can be composed of pyrrole and N-PEG200-pyrrole monomers at a ratio of 9:1, the second layer can be a mixture of pyrrole:N-alkylpaclitaxel-pyrrole at a ratio of 6:4, and the third layer can be a pyrrole:N-PEG2000-pyrrole mixture at a ratio of 9:1. This type of multilayer coating provides a release of paclitaxel over time, which is controlled by the cleavage of the agent from the pyrrole unit in the polymer and diffusion through the outer layer which also serves as passive protection from tissue and body fluids.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An article-of-manufacture comprising:
an object having a conductive surface;
an electropolymerized polymer being attached to said surface; and
at least one active substance being attached to said electropolymerized polymer, provided that said active substance is attached to said polymer via an interaction other than an electrostatic interaction or a covalent interaction.

2. The article-of-manufacture of claim 1, wherein said object is an implantable device.

3. The article-of-manufacture of claim 1, wherein said at least one active substance is selected from the group consisting of a bioactive agent, a protecting agent, a polymer having a bioactive agent attached thereto, a plurality of microparticles and/or nanoparticles having a bioactive agent attached thereto, and any combination thereof.

4. The article-of-manufacture of claim 3, wherein said protecting agent is selected from the group consisting of a hydrophobic polymer, an amphiphilic polymer, a plurality of hydrophobic microparticles and/or nanoparticles, a plurality of amphiphilic microparticles and/or nanoparticles and any combination thereof.

5. The article-of-manufacture of claim 3, wherein said bioactive agent is selected from the group consisting of a therapeutically active agent, a labeled agent and any combination thereof.

6. The article-of-manufacture of claim 1, wherein said active substance is attached to said electropolymerized polymer via an interaction selected from the group consisting of a hydrogen bond, a Van der Waals interaction, a hydrophobic interaction, a surface interaction, a physical interaction and any combination thereof.

7. The article-of-manufacture of claim 1, wherein said active substance is swelled, absorbed, embedded and/or entrapped within said electropolymerized polymer.

8. The article-of-manufacture of claim 1, wherein said electropolymerized polymer is selected from the group consisting of polypyrrole, polythienyl, polyfuranyl, a derivative thereof and any mixture thereof.

9. The article-of-manufacture of claim 1, further comprising at least one additional polymer attached to said electropolymerized polymer.

10. The article-of-manufacture of claim 9, wherein said additional polymer is selected from the group consisting of an electropolymerized polymer and a chemically-polymerized polymer.

11. The article-of-manufacture of claim 10, wherein said chemically-polymerized polymer is swelled, absorbed or embedded within said electropolymerized monomer.

12. The article-of-manufacture of claim 10, wherein said additional polymer forms a part of said electropolymerized polymer.

13. The article-of-manufacture of claim 9, wherein said active substance is further attached to said additional polymer.

14. The article-of-manufacture of claim 9, wherein said active substance is attached to said electropolymerized polymer via said additional polymer.

15. The article-of-manufacture of claim 9, wherein said additional polymer is selected from the group consisting of a hydrophobic polymer, a biodegradable polymer, a non-degradable polymer, a hemocompatible polymer, a biocompatible polymer, a polymer in which said active substance is soluble, a flexible polymer and any combination thereof.

16. The article-of-manufacture of claim 14, wherein said at least one additional polymer having said active substance attached thereto forms a part of said electropolymerized polymer.

17. The article-of-manufacture of claim 14, wherein said active substance is swelled, absorbed, embedded and/or entrapped within said additional polymer.

18. The article-of-manufacture of claim 1, being designed capable of controllably releasing said active substance in the body.

19. The article-of-manufacture of claim 18, wherein said releasing is effected during a time period that ranges from about 1 day to about 200 days.

20. The article-of-manufacture of claim 1, wherein said electropolymerized polymer has a thickness that ranges between 0.1 micron and 10 microns.

21. The article-of-manufacture of claim 1, wherein an amount of said active substance ranges from about 0.1 weight percents to about 50 weight percents of the total weight of the polymer.

22. The article-of-manufacture of claim 21, wherein an amount of said active substance is about 50 weight percents of the total weight of the polymer.

23. A process of preparing the article-of-manufacture of claim 1, the process comprising:
providing said object having said conductive surface;
providing a first electropolymerizable monomer;
providing said active substance;
electropolymerizing said electropolymerizable monomer, to thereby obtain said object having said electropolymerized polymer attached to at least a portion of a surface thereof; and
attaching said active substance to said electropolymerized polymer.

24. The process of claim 23, wherein said active substance is swelled, absorbed, embedded and/or entrapped within said electropolymerized polymer.

25. The process of claim 24, wherein attaching said active substance is performed by:
providing a solution containing said active substance; and
contacting said object having said electropolymerized polymer attached to at least a portion of a surface thereof with said solution.

26. The process of claim 23, wherein said article-of-manufacture further comprises at least one additional polymer attached to said electropolymerized polymer, said process further comprising:
attaching said additional polymer to said electropolymerized polymer, to thereby provide an object having an electropolymerized polymer onto at least a portion of a surface thereof and an additional polymer attached to said electropolymerized polymer.

27. The process of claim 26, wherein said additional polymer is an electropolymerized polymer and said process further comprising:
providing a second electropolymerizable monomer; and
electropolymerizing said second electropolymerizable monomer onto said object having said electropolymerized polymer onto at least a portion of a surface thereof.

28. The process of claim 26, wherein said additional polymer is a chemically-polymerized polymer that is swelled, absorbed or embedded within said electropolymerized monomer, and the process further comprising:
providing a solution containing said chemically-polymerized polymer; and
contacting said object having said electropolymerized polymer attached to said surface with said solution.

29. The process of claim 26, wherein said additional polymer is a chemically-polymerized polymer that is swelled, absorbed or embedded within said electropolymerized monomer, and the process further comprising:
providing a solution containing a monomer of said chemically-polymerized polymer; and
polymerizing said monomer while contacting said object having said electropolymerized polymer attached to said surface with said solution.

30. The process of claim 26, wherein said additional polymer is a chemically-polymerized polymer that forms a part of said electropolymerized polymer and providing said first electropolymerizable monomer comprises providing a first electropolymerizable monomer having a functional group capable of interacting with or forming said additional polymer.

31. The process of claim 30, wherein said functional group is selected capable of forming said additional polymer, the process further comprising: subjecting said object having said electropolymerized polymer attached thereto to a chemical polymerization of said functional group.

32. The process of claim 30, wherein said functional group is selected capable of participating is the formation of said additional polymer and the process further comprising:
providing a solution containing a substance capable of forming said additional polymer; and
contacting said object having said electropolymerized polymer attached to said surface with said solution.

33. The process of claim 32, wherein said functional group is selected from the group consisting of a photoreactive group, a cross-linking group and a polymerization-initiating group.

34. The process of claim 23, wherein said electropolymerizable monomer and/or said electropolymerizing is selected so as to provide an electropolymerized polymer having a thickness that ranges between 0.1 micron and 10 microns.

35. The process of claim 34, wherein said electropolymerizable monomer is an N-alkyl pyrrole derivative in which said alkyl has at least 3 carbon atoms.

36. The process of claim 23, further comprising, prior to said electropolymerizing, treating said surface of said object so as to enhance the adhesion of said electropolymerized polymer to said surface.

37. An electropolymerizable monomer having at least one functional group selected from the group consisting of:
(i) a functional group capable of enhancing an adhesion of an electropolymerized polymer formed from the electropolymerizable monomer to a conductive surface;
(ii) a functional group capable of enhancing absorption, swelling or embedding of an active substance within an electropolymerized polymer formed from the electropolymerizable monomer;
(iii) a functional group capable of forming a chemically-polymerized polymer;
(iv) a functional group capable of participating in the formation of a chemically-polymerized polymer;
(v) a functional group capable of providing an electropolymerized polymer formed from the electropolymerizable monomer having a thickness that ranges from about 0.1 micron to about 10 microns; and
(vi) a functional group capable of enhancing the flexibility of an electropolymerized polymer formed from the electropolymerizable monomer.

38. An electropolymerizable monomer comprising at least two electropolymerizable moieties being linked to one another.

39. The electropolymerizable monomer of claim 38, wherein said at least two electropolymerizable moieties are linked to one another via a covalent bond, a spacer or a combination thereof.

40. The electropolymerizable monomer of claim 39, wherein said spacer is selected from the group consisting of a substituted or unsubstituted alkyl, a substituted or unsubstituted alkenyl, a substituted or unsubstituted alkynyl, a substituted or unsubstituted cycloalkyl, and a substituted or unsubstituted polyalkylene glycol.

41. The electropolymerizable monomer of claim 38, wherein each of said electropolymerizable moieties is independently selected from the group consisting of substituted or unsubstituted pyrrole, thienyl and furanyl.

42. The electropolymerizable monomer of claim 38, being selected from the group consisting of 1,2,6-tri(N-propanoyl pyrrole)-hexane, 1,1′,1″,1′″-tetra(N-propanoyl pyrrole)-methane, bis-pyrrole-PEG, 1,1′-di(2-thienyl)ethylene, 3-dimethylamino-1-(2-thienyl)-propanone, 1,4-di(2-thienyl)-1,4-butandiol, and 1,2-di(2-pyrrolyl)-ethene.

43. An electropolymerizable monomer comprising at least one electropolymerizable moiety and at least one functional group capable of forming a chemically-polymerized polymer being attached to said electropolymerizable moiety.

44. An electropolymerizable monomer comprising at least one electropolymerizable moiety and at least one functional group capable of participating in the formation of a chemically-polymerized polymer being attached to said electropolymerizable moiety.

45. A method of treating a conductive surface so as to enhance the adhesion of an electropolymerized polymer to the surface, the method comprising subjecting the surface, prior to forming said electropolymerized polymer thereon, to at least one procedure selected from the group consisting of manually polishing the surface, contacting the surface with an acid, subjecting the surface to sonication and any combination thereof.

46. The method of claim 45, wherein said sonication is performed in the presence of carborundum.

47. A device for holding a medical device while being subjected to electropolymerization onto a surface thereof, the device comprising a perforated encapsulation, adapted to receive the medical device, and at least two cups adapted for enabling electrode structures to engage with said perforated encapsulation hence to generate an electric field within said perforated encapsulation.

48. A cartridge, comprising a plurality of holding devices according to claim 47, and a cartridge body adapted for enabling said plurality of holding devices to be mounted onto said cartridge body.

49. A system for coating at least one medical device, the system comprising in operative arrangement, at least one holding device according to claim 47, a conveyer and a plurality of treating baths arranged along said conveyer, wherein said conveyer is designed and constructed to convey said at least one holding device such that said at least one holding device is placed within each of said plurality of treating baths for a predetermined time period and in a predetermined order.
